(19) United States
(12) Patent Application Publication
(10) Pub. No.: US 2026/0357254 A1
(43) Pub. Date: Nov. 12, 2026

(54) COMPOSITIONS AND METHODS FOR ANTI-SENSE OLIGONUCLEOTIDE (ASO) TREATMENT OF HUNTINGTON'S DISEASE

(71) Applicant: OPHIDION INC., Bethlehem, PA (US)

(72) Inventors: Douglas J. MacNeil, Westfield, NJ (US); Brigitte Angenieux, Long Beach, CA (US); Yacoub Habib, Miami, FL (US)

(21) Appl. No.: 19/123,719

(22) PCT Filed: Apr. 3, 2023
(86) PCT No.: PCT/US2023/017275

§ 371 Date: Apr. 23, 2025

Related U.S. Application Data
(60) Provisional application No. 63/418,945, filed on Oct. 24, 2022.

(51) Int. Cl. C12N 15/113; A61K 47/64

(52) U.S. Cl. CPC C12N 15/113; A61K 47/64; C12N 2310/11; C12N 2310/315; C12N 2310/321; C12N 2310/341; C12N 2310/344; C12N 2310/3513; C12N 2320/30

(57) ABSTRACT

A composition for treating Huntington's disease (HD) by reducing endogenous Huntingtin (HTT) levels in a target includes an ASO comprising an ASO sequence selected from SEQ ID NOs: 18 to 86, and a carrier peptide comprising a peptide sequence of SEQ ID NO:1. The carrier peptide may target the nicotinic acetylcholine receptor (nAChR) of neuronal cells in order to deliver the ASO across the blood-brain barrier (BBB). In some embodiments, the ASO may be conjugated with the carrier peptide. A method of reducing endogenous HTT levels to treat HD in the target may include administering a composition including an ASO sequence selected from SEQ ID NOs: 18 to 86 to the target.

### CROSS-REFERENCE TO RELATED APPLICATIONS

The present application is a U.S. National Phase Patent Application of, and claims priority to International Application Number PCT/US2023/017275, filed on Apr. 3, 2023, which claims priority to and the benefit of U.S. Provisional Patent Application Ser. No. 63/418,945, filed Oct. 24, 2022, the entire contents of all of which are incorporated herein by reference.

### REFERENCE TO SEQUENCE LISTING

A Sequence Listing submitted as an ST.26 standards compliant XML file via Patent Center was submitted as part of the International Application and is hereby incorporated herein by reference. The file name of the XML file for the Sequence Listing is 207286WO_409472-00038_SL.xml, the date of creation of the XML file is May 11, 2023, and the size of the XML file is 228 kilobytes.

### BACKGROUND

Huntington's disease (HD) is a progressive and fatal neurodegenerative disease associated with emotional disturbances, uncontrolled movements (chorea), and cognitive decline. Certain destabilizing mutations in the human Huntington gene, which encodes for the Huntingtin (HTT) protein, have been implicated in development of HD.

While palliative therapies are available to help manage and reduce the severity of certain symptoms of HD, to this date, no effective therapies have been developed to treat the root cause of the disease or to alleviate cognitive decline.

Anti-sense oligonucleotides (ASO) can be used to effectively silence or modulate genes and repress protein translation. As such, ASOs targeted to knockdown (e.g., reduction) or knockout (e.g., substantially complete reduction) of mutated HTT would be of interest as a potential biopharmaceutical treatment for HD. However, therapeutic use of ASOs has been limited by poor bioavailability, non-specific targeting, and immunogenic effects. Design of suitable ASO sequences and delivery of such ASO sequences to intracellular targets, particularly those in the central nervous system (CNS), remain challenging.

### SUMMARY

One or more aspects of embodiments of the present disclosure are directed toward anti-sense oligonucleotide (ASO) compositions and methods for reducing endogenous Huntingtin (HTT) mRNA and protein levels in a target, for example, to treat Huntington's disease (HD). In some embodiments, the ASO compositions are complexed with a carrier to facilitate intravascular administration and delivery to targets across the blood brain barrier (BBB).

One or more embodiments of the present disclosure provide a composition for treating Huntington's disease (HD) by reducing endogenous Huntingtin (HTT) protein and/or mRNA levels in a target, the composition including: an anti-sense oligonucleotide (ASO) including an ASO sequence selected from SEQ ID NOs: 18 to 86 or a variant thereof; and a carrier peptide including a base peptide sequence of SEQ ID NO:1 or a variant thereof.

In some embodiments, the ASO sequence includes a common sub-sequence selected from SEQ ID NOs: 18 to 28.

In some embodiments, the ASO sequence is a 20-mer selected from SEQ ID NOs: 29 to 125, for example SEQ ID NOs: 29 to 86. In some embodiments, the ASO sequence is selected from SEQ ID NOs: 87, 97, 99, 100, 125 and 41.

In some embodiments, the ASO sequence includes one or more chemical modifications selected from 2′-O-methylation of A, G, C, and/or U, and substitution of phosphorothioate in a phosphodiester backbone unit. In some embodiments, the ASO sequence is 2′-O-methylated at positions 1-5 and 16-20, and phosphorothioated between positions 1-3, 6-16, and 18-20.

In some embodiments, the carrier peptide further includes a linker sequence of SEQ ID NO:14 prepended to the base sequence. In some embodiments, the carrier peptide is selected from SEQ ID NOs: 16 and 17. In some embodiments, the ASO is conjugated with the carrier peptide. In some embodiments, a ratio of the ASO and the carrier peptide is 1:5 to 1:25.

One or more embodiments of the present disclosure provide a method of treating Huntington's disease (HD) by reducing endogenous Huntingtin (HTT) protein and/or mRNA levels in a target, the method including: administering a composition including an ASO sequence selected from SEQ ID NOs: 18 to 86.

In some embodiments, the ASO sequence includes a common sub-sequence selected from SEQ ID NOs: 18 to 28.

In some embodiments, the ASO sequence includes a 20-mer selected from SEQ ID NOs: 29 to 125, for example SEQ ID NOs: 29 to 86. In some embodiments, the ASO sequence is selected from SEQ ID NOs: 87, 97, 99, 100, 125 and 41.

In some embodiments, the ASO sequence includes one or more chemical modifications selected from 2′-O-methylation of A, G, and/or U; and substitution of phosphorothioate in a phosphodiester backbone unit. In some embodiments, the ASO sequence is 2′-O-methylated at positions 1-5 and 16-20, and phosphorothioated between positions 1-3, 6-16, and 18-20.

One or more embodiments of the present disclosure provide a method of treating Huntington's disease (HD) by reducing endogenous Huntingtin (HTT) protein and/or HTT mRNA levels in a target, the method including: administering the composition of any one of claims 1-6 to the target.

In some embodiments, the composition is prepared by mixing the ASO or the variant thereof and the carrier peptide or the variant thereof in a ratio of 1:5 to 1:25 in a solution that is acceptable for parenteral administration. In some embodiments, the solution is a saline solution including 0.1 wt % to 1 wt % PEG 8000. In some embodiments, the solution is a dextrose solution including 0.01 wt % to 1.5 wt % Polysorbate 80.

In some embodiments, the administering of the composition occurs in a human subject. In some embodiments, the target is a cell, and the cell is selected from the group consisting of neurons, neuronal cells, brain cells, glial cells, astrocytes, neuronal supporting cells, and cells of the central nervous system (CNS).

In some embodiments, the method further includes conjugating the ASO to the carrier peptide, prior to administering the composition. In some embodiments, the carrier peptide further includes a linker sequence of SEQ ID NO:14 prepended to the base sequence, and the ASO is conjugated with the carrier peptide via the linker sequence.

In some embodiments, the HTT mRNA levels are decreased by greater than 20%, for example, greater than 25%, or 20% to 90%.

One or more embodiments of the present disclosure provide a composition for treating Huntington's disease (HD) by reducing endogenous Huntingtin (HTT) protein and/or mRNA levels in a target, the composition including: a 16 to 27-mer anti-sense oligonucleotide (ASO) including one of SEQ ID NOs: 18 to 28; and a carrier peptide including a base peptide sequence of SEQ ID NO:1 or a variant thereof.

The 16 to 27-mer ASO is complementary to a HTT mRNA sequence. In some embodiments, the ASO is an 18 to 24-mer. In some embodiments, the ASO is a 20-mer.

In some embodiments, the ASO sequence includes one or more chemical modifications selected from 2′-O-methylation of A, G, C, and/or U, and substitution of phosphorothioate in a phosphodiester backbone unit.

In some embodiments, the carrier peptide further includes a linker sequence of SEQ ID NO:14, prepended to the base sequence. In some embodiments, the carrier peptide is selected from SEQ ID NOs: 16 and 17. In some embodiments, the ASO is conjugated with the carrier peptide via the linker sequence.

### BRIEF DESCRIPTION OF THE DRAWINGS

FIG. 1 is a flowchart diagram summarizing the in silico procedure used to design and select ASO sequences for Huntingtin (HTT) knockdown.

FIG. 2 is a bar chart summarizing the change in measured mRNA levels in SHSY5Y cells treated with Peptide 1 (SEQ ID NO:16):ASO or Peptide 4 (SEQ ID NO: 17):ASO conjugates (ASOs 1 to 5, SEQ ID NOS: 87, 97, 99, 100, and 125), as normalized against the change in HTT1 mRNA in a DMEMF12 growth media-only control.

FIG. 3 is a bar chart summarizing the fold-change in measured mRNA levels in SHSY5Y cells treated with Peptide 1 (SEQ ID NO:16):ASO conjugates (ASOs 6, 3 and 2, SEQ ID NOs: 41, 99 and 97), as normalized against synuclein alpha (SNCA) mRNA levels and relative to a dextrose-0.6% only control.

FIG. 4 is a bar chart summarizing the fold-change in measured HTT mRNA expression levels in the hippocampus (HP) and striatum (ST) of wild-type C57bl6 mice treated with Peptide 4 (SEQ ID NO:17):ASO3 (SEQ ID NO:99) complexes, as normalized against synuclein alpha (SNCA) mRNA levels and relative to a saline control.

FIG. 5A is a graph of Open Field Total Rearing showing total rearing bouts in the three groups of mice studied in Example 4. N values represent the number of animals at the time of open field assessment. Statistics showing the total rearing bouts for the three groups of mice studied in Example 4.

FIG. 5B is a graph of Open Field Total Distance Traveled, showing the total distance for the three groups of mice studied in Example 4. N values represent the number of animals at the time of open field assessment. Statistics showing the total distance traveled for the three groups of mice studied in Example 4.

FIG. 6A is a graph comparing the soluble expanded mHTT levels in the cortex (CTX) according to Assay 6 (2B7/MW1-ST) of the vehicle control, and ASO6 mice groups studied in Example 4.

FIG. 6B is a graph comparing the aggregated mHTT levels in the cortex (CTX) according to Assay 45 (MW8/4C9-ST) of the vehicle control, and ASO6 mice groups studied in Example 4.

FIG. 6C is a graph comparing the expanded mHTT levels in the striatum (ST) according to Assay 6 (2B7/MW1-ST) of the vehicle control, and ASO6 mice groups studied in Example 4.

FIG. 6D is a graph comparing the aggregated mHTT levels in the striatum (ST) according to Assay 45 (MW8/4C9-ST) of the vehicle control, and ASO6 mice groups studied in Example 4.

FIG. 7A is a graph of Open Field Total Rearing showing total rearing bouts in the three groups of mice studied in Example 5. N values represent the number of animals at the time of open field assessment. Statistics showing the total rearing bouts for the three groups of mice studied in Example 5.

FIG. 7B is a graph of Open Field Statistics Total Distance Traveled showing total distance traveled in the three groups of mice studied in Example 5. N values represent the number of animals at the time of open field assessment. Statistics showing the total distance traveled for the three groups of mice studied in Example 5.

FIG. 8A is a graph comparing the soluble expanded mHTT levels in the cortex (CTX) according to Assay 6 (2B7/MW1-ST) of the vehicle control, and ASO6 mice groups studied in Example 5.

FIG. 8B is a graph comparing the aggregated mHTT levels in the cortex (CTX) according to Assay 45 (MW8/4C9-ST) of the vehicle control, and ASO6 mice groups studied in Example 5.

FIG. 8C is a graph comparing the soluble expanded mHTT levels in the striatum (ST) according to Assay 6 (2B7/MW1-ST) of the vehicle control, and ASO6 mice groups studied in Example 5.

FIG. 8D is a graph comparing the aggregated mHTT levels in the striatum (ST) according to Assay 45 (MW8/4C9-ST) of the vehicle control, and ASO6 mice groups studied in Example 5.

FIG. 9 is a side-by-side comparison of the Total Rearing Frequency graph of FIG. 5A and Total Distance Traveled graph of FIG. 5B with the Total Rearing Frequency graph of FIG. 7A and the Total Distance Traveled graph of FIG. 7A, providing a comparison of these functional measures between the 25 μg dose of Example 4 and the 2.5 μg dose of Example 5.

### DETAILED DESCRIPTION

### Selected Definitions

Abbreviations for nucleic acids and associated structures are used throughout this disclosure and follow the standard IUPAC nomenclature known in the art. For example, as would be understood by those of ordinary skill in the art, Adenine is Ade or A, Cytosine is Cyt or C, Guanine is Gua or G, Thymine is Thy or T, and Uracil is Ura or U.

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

As used herein, the term “carrier peptides” may refer to various peptides, including those described by SEQ ID NOS:16 and 17, combinations of one of SEQ ID NOS:1 to 7 and 9 to 13 with one of SEQ ID NOS:14 and 15, and variants thereof, that can be used as targeting or “carrier” molecules to facilitate delivery of an active molecule or effector agent to a target in the brain. For example, delivery of an effector agent to a target may be limited when the effector agent is administered alone, but may be increased in the presence of the carrier peptide, for example when the effector agent is conjugated or complexed with the carrier peptide. It will be understood that alternative carrier peptides or carrier molecules may be substituted for the specifically disclosed peptide sequences to thereby provide additional embodiments.

As used herein, the term “effector agent” refers to any molecule that imparts an effect on a target inside the blood brain barrier (BBB), for example, within tissues of the central nervous system (CNS) or brain or spinal column. The effector agent may include an ASO sequence, including those ASO sequences described herein and variants thereof. In some embodiments, the effector agent may further include, for example: short hairpin or stem loop RNA (shRNA); microRNA, double stranded RNA (dsRNA); strand template RNA (stRNA); oligonucleotides (DNA or RNA); modified oligonucleotides (DNA or RNA); aptamers; analogs and combinations of DNA and RNA; antisense oligomers (ASO); triplex-forming oligonucleotides (TFO); genes; peptides including antibody and antigen fragments; proteins, including antibodies and antigens; small chemical molecules; large chemical molecules; viral particles; liposomes; endosomes; exosomes; nanoparticles; dendrimers such as poly(amidoamine) (PAMAM); positron emission tomography (PET) ligands; eukaryotic cells; prokaryotic cells; microspheres; nanogels; and/or bionanocapsules. The effector agent can be conjugated or linked to a carrier, such as a carrier peptide or carrier peptide variant.

As used herein, “antisense oligonucleotide (ASO)” and “antisense therapeutic” are used interchangeably to refer to a non-coding, single stranded oligonucleotide sequence, typically 16-27 (or e.g., 18-22) nucleotides in length. The ASO sequence is selected to be complementary to and capable of binding or hybridizing with a target mRNA sequence. Once delivered to a suitable target (e.g., once co-localized with the target mRNA sequence), the hybridization of the ASO with the mRNA may trigger cleavage of the mRNA via one or more cellular mechanisms. For example, an ASO based on single stranded DNA (ssDNA) may activate one or more RNAase H enzymes, while an ASO based on single stranded RNA (ssRNA) may activate the RNA-induced silencing complex (RISC). Once the mRNA is cleaved by either type of enzyme, the mRNA can no longer be translated into protein. References to ASOs and/or ASO variants will be understood to encompass DNA-based as well as RNA-based counterparts or equivalents, unless otherwise clear from the context. Further, the terms explicitly include ASO molecules that have been chemically modified as described below, for example, to increase their stability and/or bioavailability, and/or to decrease their immunostimulatory effects.

As used herein, “triplex-forming oligonucleotides (TFO)” and “triplex-forming oligonucleotide therapeutics” refer to a non-coding, single stranded oligonucleotide sequence, typically 10-30 nucleotides in length, or a mimic thereof, such as peptide nucleic acids (PNAs) and/or polyamides. The oligonucleotide sequence or TFO is selected to be capable of binding within the major groove of a target genomic DNA sequence to thereby form a triple helix (triplex) structure. When binding of the TFO outcompetes binding of transcription factors or polymerases to the DNA (e.g., within regulatory sequences or coding sequences, respectively), gene transcription can be controlled or blocked. Additional information about TFOs is disclosed in e.g., Ohkubo et al., “Synthesis and triplex-forming properties of oligonucleotides capable of recognizing corresponding DNA duplexes containing four base pairs,” Nucl. Acids Res., 2015, 43(12), 5675-5686, and Jain et al, “DNA Triple Helices: biological consequences and therapeutic potential,” Biochimie. 2008, 90(8), 1117-1130, the entire content of each of which is incorporated herein by reference.

As used herein, a “fragment” of a polynucleotide or polypeptide refers to a smaller set (e.g., a contiguous sub-set) of nucleotides or peptides with respect to the referenced polynucleotide or polypeptide. The length of the fragment may range from two to N−1 nucleotides or peptides (where N is the number of residues in the referenced parent), unless further specified. For example, a fragment of a carrier peptide (described below) may refer to a peptide including any sub-sequence or truncation of the reference carrier peptide. In some embodiments, the fragment may retain at least about 80%, 85%, 90%, 95%, or 99% of the same biochemical functions as the reference. The peptide fragment may be identical to the corresponding sub-sequence in the reference carrier peptide, or may further include additional amino acid mutations or chemical derivatizations (e.g., may also be a mutant or a chemical derivative), as described herein.

As used herein, a “mutant” of a peptide (e.g., a polypeptide) refers to a polypeptide that includes one or more point mutations (amino acid mutations, e.g., residue additions, substitutions, and/or deletions) compared to a reference polypeptide, but still achieves similar biological functions. For example, a “mutant” of a polypeptide or carrier peptide may have a sequence and/or structural homology of at least about 80%, 85%, 90%, 95%, or 99% to the reference polypeptide; and may retain at least about 80%, 85%, 90%, 95%, or 99% of the same biochemical functions as the reference. The term “amino acid mutations” refers to one or more residue additions, substitutions, and/or deletions with respect to a reference polypeptide.

As used herein, a “chemical derivative” of a peptide (e.g., a polypeptide) is a modification of a referenced peptide to include a chemical modification at one or more positions, for example, to improve stability, mediate intermolecular interactions, facilitate visual localization, etc. The modification may include replacement of standard amino acids with non-standard residues and/or addition of new functional groups (e.g., methylation at a hydroxyl group). Non-limiting examples of chemical modifications of peptides include the products of techniques such as ubiquitination, methylation, fluorination, fluorescent labeling, phosphorescent labeling, and PEGylation (e.g., derivatization with ubiquitin, methyl, fluorine, a fluorescent dye, a phosphorescent dye, and polyethylene glycol, respectively). The point(s) of modification (e.g., position or starting functional group) is (are) not particularly limited, and for example, may include the N- and C-termini and suitable amino acid side chain groups. The term “chemical derivatizations” refers to one or more of the above-described chemical modifications.

As used herein, a “chemical derivative” of a polynucleotide is a modification of a referenced polynucleotide to include a chemical modification at one or more positions, for example, to improve stability, mediate intermolecular interactions, facilitate visual localization, etc. The modification may include replacement of reference functional groups (e.g., replacement of a phosphodiester with a phosphorothioate) and/or addition of new functional groups (e.g., methylation at a hydroxyl group). The point(s) of modification is (are) not particularly limited, and for example, may include the phosphates and ribose hydroxides of the backbone, and the aromatic rings of the nucleobases. Examples of such chemical modifications include moieties added to the 2′ position of one or more ribose (sugar) units; substitution or modification of internucleotide phosphodiesters in the backbone with phosphorothioates, phosphorodithioates, triazoles, amides, boranophosphates, etc.; nucleobase modifications to incorporate fluorescent groups or replace standard bases with difluorotoluene, dichlorobenzene, halogenated variants, etc.; and modification of terminus or conjugate groups with aromatic compounds, PEG, triphosphate groups, etc. Selected example modifications are disclosed in Selvam et al., “Therapeutic potential of chemically modified siRNA: Recent trends,” Chem. Biol. Drug. Des. 2017, 90(5): 665-678, and in Deleavey et al., “Designing Chemically Modified Oligonucleotides for Targeted Gene Silencing,” Chemistry & Biology, 2012, 19, 937-954, the entire content of each of which is incorporated herein by reference and attached hereto as Appendices A and B. In particular, the chemical modifications described in the context of siRNA may be applied to other types of nucleotides.

A molecule may be described as being a “functional equivalent” of another molecule if both molecules possess a similar biological activity (e.g., fulfill at least about 80%, 85%, 90%, 95%, or 99% of the same biological or biochemical role(s)), and do so at similar levels (e.g., the functional equivalent may possess at least about 80%, 85%, 90%, 95%, or 99% of the biochemical activity of the reference, and in some embodiments greater than 100% of the biochemical activity of the reference). The molecule may possess a similar activity as quantified above and be considered a functional equivalent as that term is used herein, even if, for example, the sequence of residues (e.g., primary structure) is not identical, or different chemical modifications are made to a shared basic sequence. However, it will be understood that a functional equivalent may be a fragment, mutant, or chemical derivative (as those terms are defined herein) of a reference, and may therefore possess certain structural similarities with respect to the reference.

As used herein, fragments, mutants, chemical derivatives, and functional equivalents (e.g., as defined above) of a carrier peptide may be collectively referred to as “carrier peptide variants,” “carrier-derived peptides,” or “Peptide n-derived peptides,” where “n” is any integer and is used to refer to a disclosed peptide having a specified sequence. Fragments, chemical derivatives, and functional equivalents (e.g., as defined above) of an ASO polynucleotide may be collectively referred to as “ASO variants” or “ASO-derived sequences.” References to a sequence identification number (SEQ ID NO) will be understood as disclosing and encompassing variants thereof, for example, variants having 80% to 99% sequence identity (e.g., homology or similarity) when compared to the named sequence, e.g., at least about 80%, 85%, 90%, 95%, or 99% sequence identity.

As used herein, “conjugated,” “linked,” and “complexed” are used interchangeably to describe a state in which two molecules are connectively joined. As used herein, the term “conjugate” or “conjugation” refers to the attachment of two or more entities to form one entity (molecule). For example, embodiments of the present disclosure provide for a composition including an ASO effector agent conjugated with a carrier peptide. The attachment can be by means of chemical modification, protein fusion, covalent bonds, or non-covalent bonds (e.g., ionic or intramolecular attractions), and in some embodiments may include the use of intermediary linkers, including peptide linkers, chemical (e.g., functional group or small molecule) linkers, or any means known to one skilled in the art. The joining can be permanent or reversible. For example, linkers can be enzymatically cleavable, acid cleavable, photocleavable, and/or heat sensitive (e.g., thermally cleavable). In some embodiments, several linkers can be included according to the desired properties of each linker and each component in the conjugate. Flexible linkers and linkers that increase the solubility of the conjugates are contemplated for use alone, or in combination with other linkers disclosed herein. Methods for conjugation are well known by persons skilled in the art. For example, peptide linkers can be attached and deployed by prepending and/or appending a DNA sequence encoding the linker to one or more proteins in the conjugate (e.g., as a recombinant protein).

As used herein, “target” refers to any cell that is intended to receive a carrier peptide and/or an effector agent. The target may be entirely within the central nervous system (CNS) tissue, which is isolated from the intervascular system by the BBB. In other embodiments, the target may be or include cells located in tissues outside the BBB.

The term “target cells” as used herein may also refer to cells expressing the alpha (α) subunit and/or the beta (β) subunit of the nicotinic acetylcholine receptor (nAChR). Carrier peptides that bind to the α-subunit of the nicotinic acetylcholine receptor, including carrier peptides, can thus be used to selectively bind target cells expressing the a subunit of the nicotinic acetylcholine receptor. Examples of cells that express the a subunit of the nicotinic acetylcholine receptor include, for example, the neurons, glial cells, and endothelial cells comprising the BBB. Target cells of the present disclosure also include cells whose endogenous milieu is separated by the BBB, for example, cells in the CNS, including brain cells, spinal cord cells, glial cells, and other cells supporting neurons, e.g., astrocytes or “nursing cells”. In some embodiments, the target cells may include any cell expressing the alpha subunit of the nicotinic acetylcholine receptor or a homologue thereof, such as but not limited to neuronal cells in a subject (i.e. in vivo), neuronal cells ex vivo, or cultured neuronal cells (i.e. in vitro) such as, for example primary neuronal cultured cells, or immortalized cell lines expressing alpha and/or beta subunits of the nicotinic acetylcholine receptor either naturally or through stable selection of transfected alpha and/or beta nicotinic acetylcholine receptor constructs. In some embodiments, the target cells are neuronal precursor or neuronal progenitor cells, such as neuronal progenitor stem cells that express an alpha subunit of the nicotinic acetylcholine receptor or a homologue thereof. In some embodiments of the present disclosure, the target is present within a subject, for example a mammalian subject, such as a human subject or patient. In alternative embodiments, the target is ex vivo, and in further embodiments, the target is in a biological sample, for example in vitro.

In some embodiments, the effector agent can be transported to various target cells or tissues. For example, the effector agent can be transported to any nerve cell, e.g., a nerve cell in the central nervous system, olfactory, or visual system. In some embodiments, the effector agent can also be transported to a neurologically related target cell or tissue, e.g., cells or tissues that interact with or are targets of the nervous system.

All nucleotides and peptides disclosed herein, including ASO sequences and carrier peptides, may be constructed by using any suitable method available in the art. For example, peptides may be synthesized using a peptide synthesizer (Applied Biosystems Model 433) or can be synthesized recombinantly by methods well known in the art, such as those described in Merrifield, “Solid Phase Synthesis,” J. Am. Chem. Soc., 1963, 83:2149-2154. Nucleotides may be synthesized using an oligonucleotide sequencer or by using in vitro transcription, PCR cassette, or expression vector methods well known in the art. The method by which a peptide or nucleotide of the present disclosure is synthesized does not limit the present disclosure.

In some embodiments, an effector agent or therapeutic agent, for example an ASO as disclosed herein, can be prepared to be delivered in a “prodrug” form. The term “prodrug” indicates a therapeutic agent that is prepared in an inactive form that is converted to an active form (i.e., drug) within the body or cells thereof by the action of endogenous enzymes or other chemicals and/or conditions.

As used herein, the term “gene” refers to a nucleic acid comprising an open reading frame encoding a polypeptide, and may be inclusive of all coding segment sequences (exons) as well as (optionally) non-coding intervening sequences (introns). For example, “gene” may refer to the coding sequence of a gene product, or the coding sequence together with the non-coding regions of the gene product, including the 5′UTR and 3′UTR regions, introns, and promoters or enhancers of the gene product. As used herein, the term “gene product(s)” may encompass the RNA (e.g., mRNA) transcribed from a gene, and/or the polypeptide (e.g., protein) produced by translation of the RNA. A “promoter” is a region of a genomic nucleic acid sequence at which initiation and rate of transcription are controlled. It can contain elements at which regulatory proteins and molecules can bind (such as RNA polymerase and other transcription factors) to initiate the transcription of a specific nucleic acid sequence. The term “enhancer” refers to a cis-acting regulatory sequence involved in the transcriptional activation of a nucleic acid sequence, e.g., to increase transcription of a particular gene product. An enhancer can function in either orientation and can be included upstream or downstream of the promoter.

Although some of the above definitions generally refer to a single-stranded molecule (oligonucleotides), in some embodiments and contexts the terms and usage may also encompass one or more additional strands that are partially, substantially, or fully complementary to the single-stranded molecule. Thus, a nucleic acid can encompass a double-stranded molecule or a double- or multi-stranded molecule that comprises one or more complementary strand(s) or “complement(s)” of a particular sequence. As used herein, a single stranded nucleic acid can be denoted by the prefix “ss”, a double stranded nucleic acid by the prefix “ds”, and a triple stranded nucleic acid by the prefix “ts.”

The terms “composition” and “pharmaceutical composition” are used interchangeably herein to refer to compositions or formulations administered for therapeutic, diagnostic, or prophylactic purposes, and which usually comprise one or more excipients, such as a pharmaceutically acceptable carrier (e.g., solvent or diluent) in the art that is suitable for administration to mammals, and for example, humans or human cells. Cells that are administered a composition as disclosed herein can be part of a living subject, animal, or human, including a transgenic animal for research purposes. The cells can also be cultured, for example as cells as part of an assay for screening potential pharmaceutical compositions. The compositions also can include stabilizers, preservatives, lubricants, and other adjuvants and excipients. For additional examples of carriers, stabilizers and adjuvants, see Genarro, A R., Remington: The Science and Practice of Pharmacy with Facts and Comparisons, 21st Ed., the entire content of which is incorporated herein by reference. Those having ordinary skill in the art are capable of choosing suitable formulations depending on the administration route, as described herein.

The term “pharmaceutically acceptable carrier” refers to any pharmaceutically acceptable means to mix and/or deliver an effector agent or pharmaceutical composition to a subject. For example, the term may refer to any pharmaceutically acceptable material, formulation, or vehicle (such as a liquid or solid filler, diluent, excipient, solvent, or encapsulating material) involved in carrying or transporting the subject agents from one organ, or portion of the body, to another organ, or portion of the body. The carrier is “acceptable” in the sense of being compatible with the other ingredients of the formulation as well as being compatible with administration to a subject, for example a human. The carrier can be in the form of a solid, semi-solid, or liquid diluent, cream, or capsule. Such pharmaceutical preparations are a further object of some embodiments of the present disclosure. The amount of active compound (effector agent) may be between 0.001-95% by weight of the preparation, for example, between 0.01-1% by weight in preparations for parenteral use.

The compositions disclosed herein can be administered by any convenient route, including sublingual, parenteral, enteral, mucosal, topical, subcutaneous, intravascular, intravenous, intraarterial, intramuscular, intraperitoneal, transdermal, rectal, vaginal, intranasal, intraocular, intraspinal, or intracerebroventricular. In some embodiments, the compositions as disclosed herein are not topically administered. In some embodiments, the delivery is by intranasal administration of the composition, especially for use in therapy of the brain and related organs (e.g., meninges and spinal cord). Along these lines, intraocular administration is also possible. In some embodiments, the delivery means is by intravenous (IV) administration of the composition, which is especially advantageous when a longer-lasting IV formulation is desired. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), the entire content of each of which is incorporated herein by reference.

The terms “parenteral administration” and “administered parenterally” as used herein refer to modes of administration other than enteral and topical administration. In some embodiments, the term refers to injection, but may include, without limitation, intravenous, intraarterial, intramuscular, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. The phrases “systemic administration,” “administered systemically,” “peripheral administration,” and “administered peripherally” as used herein refer to administration of an effector agent, drug, or other material other than directly into the central nervous system, for example, by subcutaneous administration, such that it enters the subject or patient's system and is subject to metabolism and other like processes.

The compositions as disclosed herein can be administered in prophylactically or therapeutically effective amounts. A “prophylactically or therapeutically effective amount” refers to an amount necessary to at least partially attain a desired effect, e.g., to delay the onset of, inhibit the progression of, or halt altogether the onset or progression of the particular disease or disorder being treated (e.g., Huntington's disease). Such amounts will depend, of course, on the particular condition being treated, the severity of the condition, and individual patient parameters including age, physical condition, size, weight, co-morbidities, and concurrent medications or treatment protocols. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a lower dose or tolerable dose can be administered for medical reasons, psychological reasons, or for virtually any other reason.

In some embodiments, the compositions disclosed herein may be administered to a patient or human subject in an amount of 0.1 mg per kg to 100 mg per kg (mpk) of subject body weight, for example, 0.1 mpk to 50 mpk, 0.2 mph to 20 mpk, 0.3 mpk to 10 mpk, or 0.5 mpk to 5 mpk. The compositions may be administered using any suitable dose volume, for example, a dose volume of 100 μL to 1000 mL, 200 μL to 750 mL, 500 μL to 500 mL, 1 mL to 200 mL, or 5 mL to 100 mL. For example, the compositions may be administered in an amount of 0.1 to 10 mpk and in a dose volume of 100 μL to 5 mL.

However, in some embodiments, the compositions disclosed herein may be particularly effective in lower doses. Without being bound by any particular theory, and without limiting the scope of the present disclosure, it is believed that since the compositions and methods disclosed herein rely on receptors, high doses of the effector agents (e.g., the anti-sense oligonucleotides) in the compositions according to embodiments of the present disclosure may—in some instances—lead to receptor down regulation and limited efficacy. Indeed, it has been surprisingly found that the conjugates, complexes and compositions according to the present disclosure can enable more effective treatment in lower doses of the effector agent (e.g., the anti-sense oligonucleotides), i.e., doses of the effector agent lower than 1,000 μg per kg (body weight), 600 μg per kg (body weight) or lower, lower than 4,000 μg per kg (body weight), or lower than 100 μg per kg (body weight).

For example, in some embodiments, the compositions disclosed herein may be administered to an animal subject (e.g., a mouse) in an amount sufficient to deliver an amount of the effector agent (e.g., anti-sense oligonucleotide) of 1 μg per kg to 4,000 μg per kg (or 0.001 mg per kg to 4 mg per kg) of subject body weight, for example, 1 μg per kg to 3,000 μg per kg, 1 μg per kg to 2,000 μg per kg, 1 μg per kg to 1,000 μg per kg, 1 μg per kg to 600 μg per kg, 2 μg per kg to 4,000 μg per kg, 2 μg per kg to 3,000 μg per kg, 2 μg per kg to 2,000 μg per kg, 2 μg per kg to 1,000 μg per kg, 2 μg per kg to 600 μg per kg, 10 μg per kg to 4,000 μg per kg, 10 μg per kg to 3,000 μg per kg, 10 μg per kg to 2,000 μg per kg, 10 μg per kg to 1,000 μg per kg, 10 μg per kg to 600 μg per kg, 20 μg per kg to 4,000 μg per kg, 20 μg per kg to 3,000 μg per kg, 20 μg per kg to 2,000 μg per kg, 20 μg per kg to 1,000 μg per kg, 20 μg per kg to 600 μg per kg, 40 μg per kg to 4,000 μg per kg, 40 μg per kg to 3,000 μg per kg, 40 μg per kg to 2,000 μg per kg, 40 μg per kg to 1,000 μg per kg, 40 μg per kg to 600 μg per kg, 100 μg per kg to 4,000 μg per kg, 100 μg per kg to 3,000 μg per kg, 100 μg per kg to 2,000 μg per kg, 100 μg per kg to 1,000 μg per kg, 100 μg per kg to 600 μg per kg, 200 μg per kg to 4,000 μg per kg, 200 μg per kg to 3,000 μg per kg, 200 μg per kg to 2,000 μg per kg, 200 μg per kg to 1,000 μg per kg, 200 μg per kg to 600 μg per kg, 350 μg per kg to 4,000 μg per kg, 350 μg per kg to 3,000 μg per kg, 350 μg per kg to 2,000 μg per kg, 350 μg per kg to 1,000 μg per kg, 350 μg per kg to 600 μg per kg, 1 μg per kg to 350 μg per kg, 2 μg per kg to 350 μg per kg, 10 μg per kg to 350 μg per kg, 20 μg per kg to 350 μg per kg, 40 μg per kg to 350 μg per kg, 100 μg per kg to 350 μg per kg, 200 μg per kg to 350 μg per kg, 1 μg per kg to 200 μg per kg, 2 μg per kg to 200 μg per kg, 10 μg per kg to 200 μg per kg, 20 μg per kg to 200 μg per kg, 40 μg per kg to 200 μg per kg, 1 μg per kg to 100 μg per kg, 2 μg per kg to 100 μg per kg, 10 μg per kg 15 to 100 μg per kg, 20 μg per kg to 100 μg per kg, 40 μg per kg to 100 μg per kg, 1 μg per kg to 50 μg per kg, 2 μg per kg to 50 μg per kg, 10 μg per kg to 50 μg per kg, 20 μg per kg to 50 μg per kg, 40 μg per kg to 50 μg per kg, 1 μg per kg to 40 μg per kg, 2 μg per kg to 40 μg per kg, 10 μg per kg to 40 μg per kg, 20 μg per kg to 40 μg per kg, 1 μg per kg to 20 μg per kg, 2 μg per kg to 20 μg per kg, 10 μg per kg to 20 μg per kg, 1 μg per kg to 10 μg per kg, 2 μg per kg to 10 μg per kg. For example, in some embodiments, the compositions disclosed herein may be administered to an animal subject (e.g., a mouse) in an amount sufficient to deliver an amount of the effector agent (e.g., anti-sense oligonucleotide) of 1 μg per kg, 2 μg per kg, 10 μg per kg, 20 μg per kg, 40 μg per kg, 100 μg per kg, 200 μg per kg, 350 μg per kg, 600 μg per kg, 1,000 μg per kg, or 2000 μg per kg. It is understood that any and all ranges between the end points and specific amounts described above are also within this disclosure, including any and all sub-ranges subsumed in the ranges above, and other ranges that may span between or across the disclosed ranges, sub-ranges or specific amounts.

Those of ordinary skill in the art would be capable of discerning the human equivalent dose from the animal (e.g., mouse) doses described above. For example, as would be understood by those of ordinary skill in the art, the same doses (per kg of body weight) described above may be used in human patients when scaling by body weight, or doses 1.7-fold higher may be used in human patients when scaling by brain weight. In some embodiments, for example, when scaling by brain weight, the compositions disclosed herein may be administered to a human subject or patient in an amount sufficient to deliver an amount of effector agent (e.g., anti-sense oligonucleotide) of 1.7 μg per kg to 6,800 μg per kg of subject body weight, for example, 1.7 μg per kg to 5,100 μg per kg, 1.7 μg per kg to 3,400 μg per kg, 1.7 μg per kg to 1,700 μg per kg, 1.7 μg per kg to 1,000 μg per kg, 3.4 μg per kg to 6,800 μg per kg, 3.4 μg per kg to 5,100 μg per kg, 3.4 μg per kg to 3,400 μg per kg, 3.4 μg per kg to 1,700 μg per kg, 3.4 μg per kg to 1,000 μg per kg, 17 μg per kg to 6,800 μg per kg, 17 μg per kg to 5,100 μg per kg, 17 μg per kg to 3,400 μg per kg, 17 μg per kg to 1,700 μg per kg, 17 μg per kg to 1,000 μg per kg, 34 μg per kg to 6,800 μg per kg, 34 μg per kg to 5,100 μg per kg, 34 μg per kg to 3,400 μg per kg, 34 μg per kg to 1,700 μg per kg, 34 μg per kg to 1,000 μg per kg, 68 μg per kg to 6,800 μg per kg, 68 μg per kg to 5,100 μg per kg, 68 μg per kg to 3,400 μg per kg, 68 μg per kg to 1,700 μg per kg, 68 μg per kg to 1,000 μg per kg, 170 μg per kg to 6,800 μg per kg, 170 μg per kg to 6,800 μg per kg, 170 μg per kg to 3,400 μg per kg, 170 μg per kg to 1,700 μg per kg, 170 μg per kg to 1,000 μg per kg, 340 μg per kg to 6,800 μg per kg, 340 μg per kg to 5,100 μg per kg, 340 μg per kg to 3,400 μg per kg, 340 μg per kg to 1,700 μg per kg, 340 μg per kg to 1,000 μg per kg, 595 μg per kg to 6,800 μg per kg, 595 μg per kg to 6,800 μg per kg, 595 μg per kg to 6,800 μg per kg, 595 μg per kg to 1,700 μg per kg, 595 μg per kg to 1,000 μg per kg, 1.7 μg per kg to 595 μg per kg, 3.4 μg per kg to 595 μg per kg, 17 μg per kg to 595 μg per kg, 34 μg per kg to 595 μg per kg, 68 μg per kg to 595 μg per kg, 170 μg per kg to 595 μg per kg, 340 μg per kg to 595 μg per kg, 1.7 μg per kg to 340 μg per kg, 3.4 μg per kg to 340 μg per kg, 17 μg per kg to 340 μg per kg, 34 μg per kg to 340 μg per kg, 68 μg per kg to 340 μg per kg, 1.7 μg per kg to 85 μg per kg, 3.4 μg per kg to 85 μg per kg, 3.4 μg per kg to 85 μg per kg, 34 μg per kg to 85 μg per kg, 68 μg per kg to 85 μg per kg, 1.7 μg per kg to 68 μg per kg, 3.4 μg per kg to 68 μg per kg, 17 μg per kg to 68 μg per kg, 34 μg per kg to 68 μg per kg, 1.7 μg per kg to 34 μg per kg, 3.4 μg per kg to 34 μg per kg, 17 μg per kg to 34 μg per kg, 1.7 μg per kg to 17 μg per kg, 3.4 μg per kg to 17 μg per kg. For example, in some embodiments, when scaling by brain weight, the compositions disclosed herein may be administered to a human subject or patient in an amount sufficient to deliver an amount of the effector agent (e.g., anti-sense oligonucleotide) of 1.7 μg per kg, 3.4 μg per kg, 17 μg per kg, 34 μg per kg, 68 μg per kg, 85 μg per kg, 170 μg per kg, 340 μg per kg, 595 μg per kg, 1,700 μg per kg, 1,000 μg per kg, or 3,400 μg per kg. It is understood that any and all ranges between the end points and specific amounts described above are also within this disclosure, including any and all sub-ranges subsumed in the ranges above, and other ranges that may span between or across the disclosed ranges, sub-ranges, or specific amounts.

As would be understood by those of ordinary skill in the art, the amount of the conjugate or complex (or composition) including the carrier peptide and the effector agent (e.g., anti-sense oligonucleotide) needed to yield these ranges of the dose of the effector agent (e.g., anti-sense oligonucleotide) will vary depending on the ratio (or relative amounts) of the carrier peptide to the effector agent (e.g., anti-sense oligonucleotide). By way of example only and without limitation, conjugates or complexes (or compositions) having ratios of effector agent (e.g., anti-sense oligonucleotide) to carrier peptide of 1:4 and 1:12, respectively, would require different total amounts of the composition as a whole to achieve the same effector agent dose. Those of ordinary skill in the art are capable of calculating the amount of the conjugate, complex or composition needed to achieve a specified effector agent dose based on the ratio of the carrier peptide to effector agent in the conjugate, complex or composition.

As used herein, the terms “administering,” “introducing,” and “providing” are used interchangeably, and according to embodiments of the present disclosure, may refer to the placement of the pharmaceutical composition including a described effector agent or ASO composition into an in vivo subject or in vitro culture by a method or route that results in at least partial localization of the agents at a desired site (e.g., target cells and/or molecules). The agents of the present disclosure can be administered by any appropriate route that results in an effective treatment in the subject.

### ASO Compositions

Huntingtin (HTT) is a protein encoded by the HTT gene in humans (NCBI Ref. NP_002102) and mice (NP_034544). Although the exact function of HTT has not yet been determined, HTT is known to be highly expressed in neuronal cells and involved in axonal transport. Further, HTT is essential for normal, successful embryonic development. The 5′ end of a normal (wild-type) HTT gene typically includes 5 to 35 CAG repeats (SEQ ID NO: 148). However, mutations in the HTT gene that result in 36 or more CAG repeats (e.g., resulting in transcription of 36 or more N-terminus Glu residues) can cause the resulting mutated HTT protein (mHTT) to be unstable and subject to enzyme cleavage, and/or have altered activity and function. Higher numbers of repeats in mHTT and higher expression of mHTT are associated with earlier onset of Huntington's disease (HD), as characterized by neuronal damage in the striatum (ST), hippocampus (HP), and cerebral cortex (CTX). The mRNA for HTT (NCBI Ref. NM_002111) includes 67 exons, with the CAG repeat region being located in Exon 1. Embodiments of the present disclosure provide for compositions including an ASO that is capable of binding to one or more of the 67 HTT mRNA exons, thereby causing knockdown of the HTT protein (or e.g., mHTT protein). The knockdown or decrease in mHTT protein may therapeutically treat or alleviate the symptoms of Huntington's disease (HD).

In some embodiments, a composition for treating Huntington's disease (HD) by reducing endogenous Huntingtin (HTT) levels includes an ASO as an effector agent, and a carrier peptide for delivery to a target. Each component is described in more detail below.

In some embodiments, the ASO may be a ssDNA sequence. Hybridization of the ssDNA to the HTT mRNA forms a DNA-RNA heteroduplex, which may then trigger cleavage of the mRNA by RNA H enzymes (including RNA H1 and RNA H2). In some embodiments, the ASO may be a ssRNA sequence. Hybridization of the ssRNA to the HTT mRNA may then trigger cleavage of the mRNA by the RNA-Induced Silencing Complex (RISC). However, embodiments of the present disclosure are not limited thereto, and may include other mechanisms of transcriptional control. It will be understood that when an embodiment is described as including an ASO with a ssDNA sequence (for example, as described in a sequence listing), an alternative embodiment including an ASO with the corresponding ssRNA sequence (i.e., with uracil residues in place of the thymine residues) is also disclosed and within the scope of the present disclosure. Similarly, when an embodiment is described as including an ASO with a ssRNA sequence (for example, as described in a sequence listing), an alternative embodiment including an ASO with the corresponding ssDNA sequence (i.e., with thymine residues in place of the uracil residues) is also disclosed and within the scope of the present disclosure.

In some embodiments, the ASO may include a combination or mixture of DNA and RNA and modified bases, e.g., as a result of being chemically modified according to the modifications described above. For example, the ASO may be a single-stranded polynucleotide including a mixture of modified RNA and DNA bases, which may facilitate improved stability and in vivo half-life. In some embodiments, the ASO may include a sequence in which a sequence of DNA bases is sandwiched between two shorter sequences of modified RNA bases, e.g., as in a “gapmer” configuration. Stated another way, the ASO may be a “gapmer” ASO including a central DNA oligomer block (“gap”), flanked by two blocks of modified RNA bases (“wings”) at the 5′- and 3′-terminal ends. The modified RNA bases in the wings may be 2′-0 modified RNA bases, which is described in more detail below. The number of bases in each section of the gapmer may be the same as in gapmers of the related art, and for example, each wing may include 2 to 7 bases or 3 to 5 bases, and the gap may include 8 to 16 bases or 10 to 12 bases. In some embodiments, for example, the ASO may be a 20-mer gapmer having a sequence including 5 modified RNA bases, 10 DNA bases, and 5 modified RNA bases, in that order. However, embodiments of the present disclosure are not limited thereto. It will be understood that any suitable number, ordering, and/or combination of RNA, DNA and modified bases may be used, and that when an embodiment is described as including an ASO with a given polynucleotide sequence, alternative embodiments including ASOs with any analogous sequence of mixed modified RNA and DNA bases are also disclosed and within the scope of the present disclosure. For example, an apparent disclosure of an ssDNA sequence (for example, as described in a sequence listing) will be understood to encompass disclosure of additional embodiments including chemical modifications of that ssDNA sequence in which one or more deoxyribonucleotide(s) is (are) replaced with the corresponding ribonucleotide(s), etc., as in the gapmer configurations.

In some embodiments, a composition for treating Huntington's disease (HD) by reducing endogenous Huntingtin (HTT) levels in a target includes an ASO and a carrier peptide, each described in more detail below.

In some embodiments, the ASO included in the ASO compositions may be 16 to 27 nucleobases in length (e.g., may be a 16-mer to 27-mer). In some embodiments, the ASO may be an 18-mer to 22-mer, or a 19-mer, 20-mer, or 21-mer. In some embodiments, the ASO may be a 20-mer.

Certain HTT mRNA sub-sequences that are as long as or longer than the ASO according to embodiments of the present disclosure (e.g., 20 bases or longer) may serve as suitable mRNA target regions for ASO-induced transcriptional arrest. Such target regions may be located within various exons of the HTT mRNA. In some embodiments, the target region within the HTT mRNA may be located within exon 2, 8-9, 12-13, 17, 27, 30, 36, 36-37, 42, 42-43, 54, 63, or 67, where the notation “i-j” indicates that the target region spans adjacent portions of the two exons, numbered i and j. Stated another way, the ASO embodiments may be capable of binding to one of the above-listed target regions.

In some embodiments, multiple ASOs according to embodiments of the present disclosure may be capable of binding within the same target region at different starting base positions. Such ASOs may thus be related to each other by a series of frame shifts along a DNA (or RNA) sequence that is complementary to the HTT mRNA target region. The related ASOs may share a common sub-sequence of between 1 to 18 nucleobases in length, for example, 6 to 16 or 8 to 14 nucleobases in length, where the common sub-sequence is included at a different starting base position (position number) in each member of the group of related ASOs. Some of the ASOs according to embodiments of the present disclosure may thus include a common sub-sequence represented by one of SEQ ID NOS: 18-28, as listed in Table 1 below, which also lists the starting position and exon of the corresponding sequence in the HTT mRNA.

TABLE 1

| SEQ | | HTT mRNA | HTT |
|---|---|---|---|
| ID | Common | Starting | mRNA |
| NO | sub-sequence | Position | Exon |
| 18 | ttcacacggtc | 437 | 2 |
| 19 | agttcataaacctggac | 1208 | 8-9 |
| 20 | aacacaatttcagaact | 1877 | 12-13 |
| 21 | tcgatgtagttcaa | 2405 | 17 |
| | ggggggtt | 3665 | 27 |
| 23 | tcattggttctcgacta | 4048 | 30 |
| 24 | ctgatggtactggatga | 4878 | 36-37 |
| 25 | acataatcacag | 5851 | 42-43 |
| 26 | aatgatggagggggtg | 8752 | 63 |
| 27 | ctggtgtctgtag | 9439 | 67 |
| 28 | ctctttactgata | 10146 | 67 |

In some embodiments, the common sub-sequence of the ASOs may be, for example, 11 to 16 nucleobases in length, and may be selected from SEQ ID NOs:18, 21, and 25-28. In some embodiments, the common sub-sequence of the ASOs may be, for example, 11 to 13 nucleobases in length, and may be selected from SEQ ID NOs:18, 25, 27, and 28.

In some embodiments, the ASO may be a 16 to 27-mer, for example, an 18 to 24-mer or a 20-mer including a common sub-sequence selected from SEQ ID NOS: 18-28. The ASO may be complementary to one HTT mRNA target region regardless of its specific length.

In some embodiments, the ASO included in the ASO compositions may be designed or selected to have a reduced risk of cross-reactivity (e.g., with non-HTT mRNA) and immunotoxicity. For example, the ASO may meet the following sequence-related criteria:
1) The ASO is complementary to an HTT mRNA exon sequence;
2) The ASO is not complementary to any mRNA sequence having one or more nucleotide polymorphisms with an occurrence frequency of >1%;
3) The ASO is not complementary to the mRNA of any human genes (e.g., any sequences in the target transcriptome) other than HTT. For example, the ASO does not appreciably bind to other mRNA sequences due to the presence of at least 3 mismatches;
4) The ASO does not have a sequence that lends itself to formation of a strong secondary structure (such as a loop or pseudoknot); and
5) The ASO does not include any known toxic motifs (e.g., motifs that trigger a hepatotoxic physiological response), such as tgc, tcc, and gc.

Stated another way, the ASO compositions according to embodiments of the present disclosure may exclude ASO sequences that: 1) are not complementary to an HTT mRNA exon sequence; 2) are complementary to any HTT mRNA sequence having one or more nucleotide polymorphisms with an occurrence frequency of greater than 1%; 3) are complementary to the mRNA of any transcriptome sequence other than HTT; 4) has a strong secondary structure; and/or 5) include any of the above-described toxic motifs.

In some embodiments, the ASO may have a total guanine and cytosine content of 20% to 60% with respect to the total number of bases, for example, 25% to 55%, 30% to 50%, or 35% to 45%.

In some embodiments, the ASO may be selected from particular 20-mer ASO sequences and variants thereof that meet the preceding combined criteria. In some embodiments, for example, the ASO may be selected from the ASO sequences listed in Table 2 below (SEQ ID NOS:29 to 86), which also lists the starting position and exon of the corresponding sequence in the HTT mRNA. In some embodiments, the ASO sequences may be selected from the Table 2 sequences and variants thereof, including sequences having 80% to 99% sequence identity thereto, e.g., at least about 80%, 85%, 90%, 95%, or 99% sequence identity thereto, etc. For example, the ASO sequences may be selected from “gapmer” modified variants of SEQ ID NOS:29 to 86, respectively referred to as SEQ ID NOS:87 to 144, also shown in Table 2.

TABLE 2

| | | HTT mRNA | HTT | SEQ | |
|---|---|---|---|---|---|
| SEQ ID | | Starting | mRNA | ID | Gapmer variant |
| NO | ASO Sequence | Position | Exon | NO | sequence |
| 29 | ttcacacggtctttcttggt | 428 | 2 | 87 | uucacacggtctttcuuggu |
| 30 | attcacacggtctttcttgg | 429 | 2 | 88 | auucacacggtctttcuugg |
| 31 | gattcacacggtctttcttg | 430 | 2 | 89 | gauucacacggtcttucuug |
| 32 | tgattcacacggtctttctt | 431 | 2 | 90 | ugauucacacggtctuucuu |
| 33 | atgattcacacggtctttct | 432 | 2 | 91 | augautcacacggtcuuucu |
| 34 | aatgattcacacggtctttc | 433 | 2 | 92 | aaugattcacacggtcuuuc |
| 35 | caatgattcacacggtcttt | 434 | 2 | 93 | caaugattcacacggucuuu |
| 36 | acaatgattcacacggtctt | 435 | 2 | 94 | acaaugattcacacggucuu |
| 37 | gacaatgattcacacggtct | 436 | 2 | 95 | gacaatgattcacacggucu |
| 38 | agacaatgattcacacggtc | 437 | 2 | 96 | agacaatgattcacacgguc |
| 39 | agttcataaacctggacaag | 1205 | 8-9 | 97 | aguucataaacctggacaag |
| 40 | cagttcataaacctggacaa | 1206 | 8-9 | 98 | caguucataaacctggacaa |
| 41 | gtcagttcataaacctggac | 1208 | 8-9 | 99 | gucagttcataaaccuggac |
| 42 | aacacaatttcagaactgtc | 1874 | 12-13 | 100 | aacacaatttcagaacuguc |
| 43 | ctaacacaatttcagaactg | 1876 | 12-13 | 101 | cuaacacaatttcagaacug |
| 44 | tctaacacaatttcagaact | 1877 | 12-13 | 102 | ucuaacacaatttcagaacu |
| 45 | tcgatgtagttcaagatgtc | 2399 | 17 | 103 | ucgaugtagttcaagauguc |
| 46 | atcgatgtagttcaagatgt | 2400 | 17 | 104 | aucgatgtagttcaagaugu |
| 47 | gatcgatgtagttcaagatg | 2401 | 17 | 105 | gaucgatgtagttcaagaug |
| 48 | tgatcgatgtagttcaagat | 2402 | 17 | 106 | ugaucgatgtagttcaagau |
| 49 | atgatcgatgtagttcaaga | 2403 | 17 | 107 | augaucgatgtagttcaaga |
| 50 | catgatcgatgtagttcaag | 2404 | 17 | 108 | caugatcgatgtagtucaag |
| 51 | ccatgatcgatgtagttcaa | 2405 | 17 | 109 | ccaugatcgatgtaguucaa |
| 52 | ggggggtttgttagagaagg | 3653 | 27 | 110 | ggggggtttgttagagaagg |
| 53 | aggggggtttgttagagaag | 3654 | 27 | 111 | aggggggtttgttagagaag |
| 54 | aaggggggtttgttagagaa | 3655 | 27 | 112 | aaggggggtttgttagagaa |
| 55 | gaaggggggtttgttagaga | 3656 | 27 | 113 | gaaggggggtttgttagaga |
| 56 | agaaggggggtttgttagag | 3657 | 27 | 114 | agaaggggggtttgtuagag |
| 57 | agagaaggggggtttgttag | 3659 | 27 | 115 | agagaaggggggtttguuag |
| 58 | tagagaaggggggtttgtta | 3660 | 27 | 116 | uagagaaggggggttuguua |
| 59 | ttagagaaggggggtttgtt | 3661 | 27 | 117 | uuagagaaggggggtuuguu |
| 60 | cttagagaaggggggtttgt | 3662 | 27 | 118 | cuuagagaagggggguuugu |
| 61 | acttagagaaggggggtttg | 3663 | 27 | 119 | acuuagagaagggggguuug |
| 62 | gacttagagaaggggggttt | 3664 | 27 | 120 | gacuuagagaagggggguuu |
| 63 | ggacttagagaaggggggtt | 3665 | 27 | 121 | ggacutagagaagggggguu |
| 64 | tcattggttctcgactaaag | 4045 | 30 | 122 | ucauuggttctcgacuaaag |
| 65 | atcattggttctcgactaaa | 4046 | 30 | 123 | aucautggttctcgacuaaa |
| 66 | catcattggttctcgactaa | 4047 | 30 | 124 | caucattggttctcgacuaa |
| 67 | ccatcattggttctcgacta | 4048 | 30 | 125 | ccaucattggttctcgacua |
| 68 | ctgatggtactggatgagtc | 4875 | 36 | 126 | cugauggtactggatgaguc |
| 69 | cacctgatggtactggatga | 4878 | 36-37 | 127 | caccugatggtactggauga |
| 70 | acataatcacagaagagaat | 5843 | 42 | 128 | acauaatcacagaagagaau |
| 71 | gacagacataatcacagaag | 5848 | 42-43 | 129 | gacagacataatcacagaag |
| 72 | ctgacagacataatcacaga | 5850 | 42-43 | 130 | cugacagacataatcacaga |
| 73 | tctgacagacataatcacag | 5851 | 42-43 | 131 | ucugacagacataatcacag |
| 74 | gtcaccaggacaccaaggag | 7556 | 54 | 132 | gucaccaggacaccaaggag |
| 75 | aatgatggagggggtggact | 8748 | 63 | 133 | aaugatggagggggtggacu |
| 76 | gtaaatgatggagggggtgg | 8751 | 63 | 134 | guaaatgatggagggggugg |
| 77 | ggtaaatgatggagggggtg | 8752 | 63 | 135 | gguaaatgatggagggggug |
| 78 | ctggtgtctgtagaagtctg | 9432 | 67 | 136 | cuggugtctgtagaagucug |
| 79 | tatctggtgtctgtagaagt | 9435 | 67 | 137 | uaucuggtgtctgtagaagu |
| 80 | ctctatctggtgtctgtaga | 9438 | 67 | 138 | cucuatctggtgtctguaga |
| 81 | cctctatctggtgtctgtag | 9439 | 67 | 139 | ccucuatctggtgtcuguag |
| 82 | ctctttactgatataattaa | 10139 | 67 | 140 | cucuutactgatataauuaa |
| 83 | tctctttactgatataatta | 10140 | 67 | 141 | ucucuttactgatataauua |
| 84 | aatctctttactgatataat | 10142 | 67 | 142 | aaucuctttactgatauaau |
| 85 | taatctctttactgatataa | 10143 | 67 | 143 | uaauctctttactgauauaa |
| 86 | aattaatctctttactgata | 10146 | 67 | 144 | aauuaatctctttacugaua |

In some embodiments, the ASO sequence is selected from SEQ ID NOs:29, 39, 41, 42, and 61 and variants thereof (including sequences having 80% to 99% sequence identity thereto, e.g., at least about 80%, 85%, 90%, 95%, or 99% sequence identity thereto, etc.), such as the corresponding gapmer variants of SEQ ID NOS: 87, 91, 99, 100, and 125. The sequences may be alternatively referred to herein by the names in Table 3:

TABLE 3

| SEQ | | | SEQ | |
|---|---|---|---|---|
| ID | | Reference | ID | Gapmer variant |
| NO | ASO Sequence | name | NO | sequence |
| 29** | ttcacacggtctttcttggt | ASO1** | 87** | uucacacggtctttcuuggu |
| 39** | agttcataaacctggacaag | ASO2** | 97** | aguucataaacctggacaag |
| 41* | gtcagttcataaacctggac | ASO3* | 99* | gucagttcataaaccuggac |
| 42** | aacacaatttcagaactgtc | ASO4** | 100** | aacacaatttcagaacuguc |
| 67** | ccatcattggttctcgacta | ASO5** | 125** | ccaucattggttctcgacua |
| 41* | gtcagttcataaacctggac | ASO6* | 99* | gucagttcataaaccuggac |

*In Table 3, ASO3 (SEQ ID NO: 99) refers to the gapmer variant sequence of ASO6 (SEQ ID NO: 41). ASO6 also has different wing modifications than those shown below with respect to wing modified SEQ ID NO. 99. Specifically, ASO6 has 2-methoxyethoxy modified RNA on the wing RNA instead of the 2-methoxy modified RNA shown below in SEQ ID NO: 145.
**In Table 3, ASO1, ASO2, ASO4 and ASO5 refer to the gapmer variant sequences SEQ ID NOs: 87, 97, 100 and 125, while the SEQ ID NOs: 29, 39, 42 and 67 are provided to show the variants.

In Tables 2 and 3, the modified RNA bases at positions 1-5 and 16-20 in the gapmer variant sequences may be 2′-O-methylated, and the bases at positions 1-3, 6-15, and 18-20 and may be connected via phosphorothioate linkages, as described in more detail in the following section.

### Chemical Modifications of ASO Sequences

It is well known that unmodified ASOs (e.g., ASO sequences consisting only of standard nucleobases) have short half-lives in vivo due to rapid degradation, which limits their utility as therapeutics. Accordingly, in some embodiments, the ASO may be chemically modified at one or more bases to provide improved in vivo stability and therapeutic efficacy. Examples of such chemical modifications include those described above in connection with the term “chemical derivatives,” but are not limited thereto.

In some embodiments, the ASO or ASO variant may be a 20-mer having a “gapmer” configuration as described above, e.g., so that the first five bases and last five bases of the sequence are RNA or modified RNA bases, and the middle (ten) bases are DNA bases.

In some embodiments, the ASO or ASO variant includes one or more chemical modifications selected from 2′-O-methylation of A, G, C, T, and/or U; and substitution of a phosphodiester backbone unit with a phosphorothioate unit. In some embodiments, the ASO includes one or more instance of both types of chemical modifications. For example, in some embodiments the 20-mer ASO may have a sequence is 2′-O-methylated at positions 1-5 and 16-20, and phosphorothioated between positions 1-3, 6-16, and 18-20. Here, the position number n refers to the nth base within the sequence starting from the 5′-end.

For example, the ASO variant may be represented by the following generic sequence showing the positions and types of chemical modifications to each strand:
mN*mN*mN mNmN N*N*N*N*N*N*N*N*N*N*mNmN mNmN*mN*

In the above sequence, mN indicates a 2′-O-methyl modified base, and * indicates a phosphorothioate linkage (e.g., in place of a standard phosphodiester linkage). It is understood that the underlying or original base sequence may be the same as any ASO sequence according to embodiments of the present disclosure, for example, as represented by any of SEQ ID NOS:29 to 86.

In some embodiments, a 20-mer ASO may be an ASO variant having a gapmer sequence of 5 RNA bases, 10 DNA bases, and 5 RNA bases, where the sequence is 2′-O-methylated at each of the RNA bases, and phosphorothioated at each of the middle DNA bases and within the 2 inter-base linkages at each end of the sequence.

For example, the ASO may be a variant of ASO3 (SEQ ID NO:99) that includes the following set of modifications:

mG*mU*mC mAmG T*T*C*A*T*A*A*A*C*C*mUmG mGmA*mC*
(wing modified SEQ ID NO 145)

wherein, in the above sequence, A, G, C, T, and U are the standard DNA and RNA bases; mG, mC, mU, mA are 2′-O-methyl modified RNA bases; and * indicates a phosphorothioate linkage (e.g., in place of a standard phosphodiester linkage). For example, the first two bases are phosphorothiate 2′-O-methyl RNA bases, the next 3 bases are phosphorodiester 2′-O-methyl RNA bases, the next 10 bases are phosphorothioate DNA bases, the next 3 bases are phosphorodiester 2′-O-methyl RNA bases, and the last two bases are phosphorothiate 2′-O-methyl RNA bases.

In some embodiments, the ASO may be a variant of any of the ASO sequences of SEQ ID NO:29 to 86, including substantially the same modifications (e.g., with respect to position within the 20-mer sequence) as detailed for SEQ ID NO:67, as represented by corresponding SEQ ID NOS:87 to 144 (e.g., including the described 2′-O-methylation and phosphorothioate modifications at the indicated positions).

In some embodiments, the ASO may be an ASO variant with fewer phosphodiester linkages. Without being bound by any particular theory, the 4× phosphodiester linkages may, in some instances, reduce the potential for toxicity, but such 4× phosphodiester linkages may, in some instances, result in a metabolically unstable ASO. In some embodiments, more stable variants may incorporate 1 to 3 phosphodiester linkages instead of 4 phosphodiester linkages.

In some embodiments, the ASO may be a variant of any of SEQ ID NOs: 29, 39, 41, and 42 including substantially the same modifications (e.g., with respect to position within the 20-mer sequence) as detailed for SEQ ID NO: 67 and its gapmer variant sequence SEQ ID NO:125 (ASO5) (for example, represented by SEQ ID NOs: 87 (ASO1), 97 (ASO2), 99 (ASO3), or 100 (ASO4). For example, the Example data presented herein were collected using ASO compositions that were chemically modified in this manner.

It will be understood that the descriptions herein of ASO compositions encompass additional ASO variant embodiments, as noted above. For example, additional embodiments may include ASO fragments, extensions (e.g., longer ASO sequences complementary to the HTT mRNA target regions), and/or functional equivalents having at least about 80%, 85%, 90%, 95%, or 99% sequence and/or structural homology to ASO sequences described herein, as well as chemical derivatives of all of the above.

### Carrier Peptide

In certain embodiments, the composition further includes a carrier peptide (e.g., carrier-derived peptide) to facilitate delivery of the ASO or another effector agent to a suitable target (e.g., a cell or tissue in which HTT is expressed). The carrier peptide may target (e.g., bind to) a receptor included on a particular cell or tissue of interest, and may subsequently facilitate passage of the ASO payload into that cell or tissue.

The carrier peptide may include or be formed of two oligomers: a base sequence; and a linker sequence. The linker sequence may facilitate conjugation of the carrier peptide to the ASO or effector agent (e.g., may be the point of contact between the effector agent and the carrier peptide), and the base sequence may facilitate delivery of the conjugate to the specific target. The C-terminus of the base sequence may be linked to the N-terminus of the linker sequence (e.g., the linker sequence is prepended to the base sequence) to form the carrier peptide.

### Base Sequence

Lynx1 is a protein that binds to neuronal nicotinic receptors (NNRs), also referred to as nicotinic acetylcholine receptors (nAChRs). Detailed information about lynx1 is disclosed in Ibanez-Tallon et al., 2002, Neuron, 33:893-903, the entire content of which is incorporated herein by reference. nAChRs are frequently expressed in target cells of the blood brain barrier (BBB), and the loop2 region of lynx1 is the putative binding domain for nAChRs. As such, lynx1-loop2-derived base sequences (e.g., base sequences derived from the loop2 domain of human lynx1, in lieu of the full lynx1 protein) can be used as carriers for targeting and delivery of a therapeutic effector agent across the BBB via the nAChRs.

Cellular uptake and transport of the effector agent across the BBB via the interaction of lynx1-loop2-derived base sequences with nAChRs can be determined by, for example, cellular imaging or confocal microscopy of relevant tissue sections. Lynx1-loop2-derived peptides are described in more detail in e.g., U.S. Pat. No. 8,629,114, issued on Jan. 14, 2014; U.S. Pat. No. 9,522,193, issued on Dec. 20, 2016; U.S. Pat. No. 9,913,915, issued on Mar. 13, 2018; U.S. Pat. No. 10,328,156, issued on Jun. 25, 2019; U.S. Pat. No. 10,772,966, issued on Sep. 15, 2020, and U.S. Ser. No. 17/020,613, filed on Sep. 14, 2020, the entire content of each of which is incorporated herein by reference. The terms “lynx1-loop2-derived peptide” and “base sequence” may be interchangeably used herein.

In some embodiments, for example, the composition may include a carrier peptide including a base sequence having the general sequence of SEQ ID NO:1, X₁-X₂-X₃-X₄-X₅-X₆-X₇-X₈-X₉-X₁₀-R-X₁₂-K-X₁₄-X₁₅-X₁₆, in which for SEQ ID NO:1:
X₁ is M or T;
X₂ is T or I;
X₃ is T or W;
X₄ is R or C;
X₅ is T or D;
X₆ is Y, I, or G;
X₇ is F or Y;
X₈ is T or C;
X₉ is P, N, or S;
X₁₀ is Y, T, or S;
X₁₂ is M or G;
X₁₄ is V or R;
X₁₅ is R, S, A, or I; and
X₁₆ is K, S, or D.

In some embodiments, the carrier peptide may include a base sequence that is similar to or conserved with a sequence in an analogue lynx1 protein of another (e.g., non-human) species. In some embodiments, for example, the base sequence may be represented by any of MTTRTYFTPYRMKVRK (SEQ ID NO:2), MTTRTYYTPTRMKVSK (SEQ ID NO:3), MTWCDYFTPSRGKVRKS (SEQ ID NO:4), or MTTRTYFTPYRGKVRK (SEQ ID NO:5). In some embodiments, the base sequence may be selected from SEQ ID NOS:2 to 5 and sequences having 80% to 99% sequence identity thereto, e.g., at least about 80%, 85%, 90%, 95%, or 99% sequence identity thereto, etc.

In some embodiments, the carrier peptide may include a lynx1-loop2-derived base sequence with the sequence MTTRTYATPYRMKVRKS (SEQ ID NO:6), which is similar to SEQ ID NO:2 except that the phenylalanine (F) at position 7 is substituted with alanine (A). In some embodiments, the carrier peptide may include a lynx1-loop2-derived base sequence with the sequence MTTRTYFTPYAMADRKS (SEQ ID NO:7), which is similar to SEQ ID NO:2 except that the RMKV residues at positions 11-14 are respectively substituted with AMAD. In some embodiments, the base sequence may be selected from SEQ ID NOS:6 and 7 and sequences having 80% to 99% sequence identity thereto, e.g., at least about 80%, 85%, 90%, 95%, or 99% sequence identity thereto, etc.

In some embodiments, additional amino acids may be added (appended, prepended, or inserted) to the lynx1-loop2-derived base sequence. For example, the sequence of MPENPRPGTP (SEQ ID NO:8) may be added to a suitable position within the 16-mer peptide of SEQ ID NO:1. In one embodiment, MPENPRPGTP (SEQ ID NO: 8) is added between residues X₃ and X₄ of SEQ ID NO:1 as defined above, resulting in X₁X₂X₃MPENPRPGTPX₄X₅X₆X₇X₈X₉X₁₀RX₁₂KX₁₄X₁₅X₁₆ (SEQ ID NO:9). In some embodiments, the carrier peptide may include a lynx1-loop2-derived base sequence with the sequence MTTMPENPRPGTPRTYFTPYRMKVRKS (SEQ ID NO:10), or a sequence having 80% to 99% sequence identity thereto, e.g., at least about 80%, 85%, 90%, 95%, or 99% sequence identity thereto, etc.

In some embodiments, the carrier peptide may include the lynx1-loop2-derived base sequence of SEQ ID NO:4.

In some embodiments, the lynx1-loop2-derived base sequence may be a 12-mer peptide (e.g. peptide fragment) having a sequence derived from SEQ ID NO:1, and for example may be SEQ ID NO:11, X₃-X₄-X₅-X₆-X₇-X₈-X₉-X₁₀-R₁₁-X₁₂-K₁₃-X₁₄, in which for SEQ ID NO:11:
X₃ is T or W;
X₄ is R or C;
X₅ is T or D;
X₆ is any amino acid;
X₇ is F or Y;
X₈ is any amino acid;
X₉ is any amino acid;
X₁₀ is any amino acid;
X₁₂ is M or G; and
X₁₄ is V or R.

In some embodiments, a SEQ ID NO:1-derived 12-mer base sequence has the sequence of SEQ ID NO:12, X₃-X₄-X₅-X₆-X₇-X₈-X₉-X₁₀-R-X₁₂-K-X₁₄, in which for SEQ ID NO:12:
X₃ is T or W;
X₄ is R or C;
X₅ is T or D;
X₆ is Y, G or I;
X₇ is F or Y;
X₈ is T or C;
X₉ is P, N, or S;
X₁₀ is Y, T, or S;
X₁₂ is M or G; and
X₁₄ is V.

In some embodiments, a SEQ ID NO:1-derived 12-mer base sequence has the sequence of SEQ ID NO:13, X₃-X₄-X₅-X₆-X₇-X₈-X₉-X₁₀-R₁₁-X₁₂-K₁₃-X₁₄, in which for SEQ ID NO:13:
X₃ is T or W;
X₄ is R or C;
X₅ is T or D;
X₆ is Y, G or I;
X₇ is F or Y;
X₈ is T;
X₉ is P;
X₁₀ is Y or T;
X₁₂ is M or G; and
X₁₄ is V.

In some embodiments, individual substitutions, deletions or additions that alter, add or delete a single amino acid or a small percentage of amino acids may be made to a lynx1-loop2-derived base sequence of the present disclosure (e.g., any of SEQ ID NOs:1 to 7 or 9 to 13). Such substitutions, insertions, and/or deletions may typically be in the range of about 1 to 5 amino acids. The choice of amino acids for conservative substitutions may be selected according to the location of the amino acid to be substituted in the peptide. For example, a conservative substitution for an amino acid on the exterior of the peptide, which is exposed to solvents and/or potential intermolecular interactions, may differ from a conservative substitution for an amino acid in the interior of the peptide, which is not exposed to solvents and intermolecular interactions. Such context-sensitive selection of conservative amino acid substitutions are well known in the art, for example as disclosed in Dordo et al, J. Mol. Biol., 1999, 217, 721-739; Taylor et al, J. Theor. Biol., 119 (1986); 205-218; and S. French and B. Robson, J. Mol. Evol., 19 (1983)171, the entire content of each of which is incorporated herein by reference. For example, conservative amino acid substitutions suitable for amino acids on the exterior of a protein or peptide (i.e. amino acids exposed to a solvent), may include the following substitutions: substitution of Y with F; T with S, K, or A; P with A; E with D or Q; N with D or G; R with K; G with N or A; T with S, K, or A; D with N or E, I with L or V, F with Y or L; S with T or A, R with K, G with N or A, K with R; A with S, K, P, G, T, or V; W with Y; and M with L. Accordingly, in some embodiments, the carrier peptide may include a lynx1-loop2-derived base sequence variant with a sequence selected from SEQ ID NOs: 1 to 7 or 9 to 13 that is modified to include one or more conservative substitutions.

However, embodiments of the present disclosure are not limited thereto, and for example, compositions according to embodiments of the present disclosure may incorporate lynx1-loop2 base sequence variants, other base sequences having no sequence relationship to the lynx1-loop2 base sequence, or other components and methods of transporting the ASO across the BBB.

### Linker Sequence

The linker sequence may include any suitable peptide sequence that is capable of mediating or facilitating an inter- or intramolecular association between the carrier peptide and the effector agent. Stated another way, the linker sequence of the carrier peptide may participate in one or more conjugate-forming interactions with the effector agent. Non-limiting examples of such interactions include electrostatic attraction, hydrogen bonding, Van der Waals interactions, covalent or ionic bond formation, and combinations thereof. Although embodiments of the present disclosure provide compositions in which the carrier peptide is mixed and conjugated with ASO as the effector agent, it will be understood that in alternative embodiments, the carrier peptide may be suitably conjugated with other effector agents, such as drugs, antibodies, and oligonucleotides.

In some embodiments, the carrier peptide may include a linker sequence including 1 to 4 C-terminal glycine residues (SEQ ID NO: 147).

In some embodiments, the carrier peptide may include a linker sequence including 1 to 4 C-terminal glycine residues (SEQ ID NO: 147) (for example, 1, 2, 3, or 4 Gly residues), followed by a 3 to 12 residue sub-sequence capable of facilitating an interaction with an effector agent. For example, because siRNA, ASO, and TFO are negatively charged, the carrier peptide residue sub-sequence may include or consist of positively charged residues so that an electrostatic attraction can be formed with the negatively charged effector agent (e.g., siRNA, ASO, or TFO).

Stated another way, in some embodiments, the effector agent (e.g., siRNA, ASO, or TFO) may be conjugated with the carrier peptide via the linker sequence due an electrostatic attraction between the positively charged linker sequence and the negatively charged effector agent.

In some embodiments, for example, at least 3 of the 3 to 12 residues may be selected from arginine and lysine, and the remaining residues may be any standard amino acid. In some embodiments, 4 to 10, or 6 to 8 residues may be selected from arginine and lysine, and the remaining residues may be any standard amino acid.

In some embodiments, the sub-sequence that is capable of facilitating an electrostatic interaction between the linker sequence and a negatively charged effector agent may include 3 to 11 amino acids, for example, 5 to 10 amino acids, or 6 to 9 amino acids as described above (e.g., such that at least 3 are selected from Arg and Lys).

For example, the linker sequence may be represented by GGGG-X₅-X₆-X₇-X₈-X₉-X₁₀-X₁₁-X₁₂-X₁₃-X₁₄-X₁₅-X₁₆ (SEQ ID NO: 14), where the glycine residues at positions 2 to 4 are optional (e.g., may be present or absent), and the amino acids of X₅ to X₁₆ are selected from the standard amino acids or are absent, provided that at least three of these residues are present and are selected from Arg and Lys. For example, the linker sequence may be represented by (G)ₙ-X₅-X₆-X₇-X₈-X₉-X₁₀-X₁₁-X₁₂-X₁₃-X₁₄-X₁₅-X₁₆ (SEQ ID NO: 149), where n is an integer of 1 to 4 and the amino acids of X₅ to X₁₆ are the same as above.

In some embodiments, the 3 to 12 residue sub-sequence may only include positively charged amino acids, and for example, all of the 3 to 12 amino acids may be independently selected from arginine and lysine. For example, in the linker sequence of SEQ ID NO: 14, the amino acids of X₅ to X₁₆ are selected from Arg and Lys or are absent, provided that at least of these three residues are present. Stated in other words, the linker sequence may be represented by (G)ₙ-(X₅)ₘ (SEQ ID NO: 146), where n is an integer of 1 to 4, each instance of X₅ is independently selected from arginine and lysine, and m is an integer of 3 to 12.

In some embodiments, the carrier peptide may include a linker sequence including 4 C-terminal glycine residues followed by a 7 to 9 residue sub-sequence capable of facilitating an electrostatic interaction between the linker sequence and the ASO (SEQ ID NO: 15). In some embodiments, the 7 to 9 amino acids may include or consist of arginine or lysine residues. And in some embodiments, the 7 to 9 amino acids may include or consist of all arginine residues, or all lysine residues.

For example, the linker sequence may be represented by GGGG-X₅-X₆-X₇-X₈-X₉-X₁₀-X₁₁-X₁₂-X₁₃ (SEQ ID NO: 15), where the amino acids of X₅ to X₁₆ are selected from Arg and Lys or are absent, provided that at least seven of these residues are present. For example, the linker sequence may be represented by (G)ₙ-(X₅)ₘ (SEQ ID NO: 150), where n is an integer of 1 to 4, each instance of X₅ is independently selected from arginine and lysine, and m is an integer of 7 to 9.

In some embodiments, the carrier peptide may include a linker sequence including 4 C-terminal glycine residues followed by 7 or 9 arginine residues (SEQ ID NO: 151). In some embodiments, the carrier peptide (which includes the base sequence and the linker sequence) may have a sequence of MTWCDYFTPSRGKVRKSGGGGRRRRRRRRR (SEQ ID NO:16). In some embodiments, the carrier peptide may have a sequence of MTWCDYFTPSRGKVRKSGGGGRRRRRRR (SEQ ID NO: 17). In some embodiments, the carrier peptide may be selected from SEQ ID NOS:16 and 17 and variant sequences having 80% to 99% sequence identity thereto, e.g., at least about 80%, 85%, 90%, 95%, or 99% sequence identity thereto, etc.

Each of the amino acids in the linker sequence may have any suitable stereochemistry. When the linker sequences according to embodiments of the present disclosure includes one or more arginine and/or lysine residues, the arginine and/or lysine residues may each independently be L- or D-residues.

In some embodiments, the linker sequence may include, consist essentially of, or consist of a poly-L-arginine peptide having 7, 8, or 9 arginine residues (SEQ ID NO: 152). In some embodiments, the linker sequence may include, consist essentially of, or consist of a poly-D-arginine peptide having 7, 8, or 9 arginine residues (SEQ ID NO: 153). It will be understood that the descriptions herein of carrier peptides encompass embodiments including carrier peptide variants as defined above. For example, additional embodiments may include carrier peptide fragments, mutants, and/or functional equivalents having at least about 80%, 85%, 90%, 95%, or 99% sequence and/or structural homology to peptide sequences described herein, as well as chemical derivatives of all of the above. Doses and Dosages

Throughout this disclosure, the terms “dose” and “dosage” are used in their art recognized senses. For example, while the term “dose” refers to a specified or measured amount of a therapeutic agent to be taken or given at any one given time, the term “dosage” refers to both the size of the dose to be taken as well as the timing regimen for taking multiple doses. Accordingly, for example, X amount of a therapeutic agent is a “dose,” while X amount of a therapeutic agent to be taken twice daily for two weeks is a “dosage.”

According to some embodiments of the present disclosure, a dosage of the conjugate, complex and/or composition may include any number of doses administered according to a dosage plan prescribed by a physician, without limitation. For example, in some embodiments, the dosage plan may include a single dose of the conjugate and/or composition administered only once. In some embodiments, however, the dosage plan may include multiple doses of the conjugate, complex and/or composition administered according to a time interval prescribed by a physician. For example, in some embodiments, the dosage plan may include periodic doses, with subsequent doses administered a prescribed interval from the first dose. The prescribed interval is not particularly limited, and may be any interval prescribed by a physician. In some embodiments, however, the prescribed interval may be 1 hour to 6 months, for example, 1 hour to 3 months, 1 hour to 1 month, 1 hour to 1 week, or 1 hour to 1 day.

Each dose of the conjugate, complex and/or composition may comprise a pharmaceutical composition including the conjugate, complex and/or composition and one or more pharmaceutically acceptable excipients, such as (but not limited to) pharmaceutically acceptable carriers. The amount of the conjugate, complex and/or composition in each dose may vary depending on the prescription of the physician. But in some embodiments, each dose includes a low dose or reduced dose of the effector agent in the conjugate, complex and/or composition compared to a conventional dose unit. Such doses are described in more detail above. By way of illustration, in some embodiments, as discussed above, each dose may include a pharmaceutical composition including the conjugate in an amount sufficient to deliver an amount of the effector agent of lower than 0.1 mg per kg of body weight of the intended subject (or recipient), for example, lower than 4,000 μg per kg body weight of the intended subject, or lower than 1,000 μg per kg body weight of the intended subject.

### Treatment Methods

A method of treating Huntington's disease (HD) by reducing endogenous Huntingtin (HTT) protein and/or mRNA levels in a target (e.g., a target cell and/or a patient), according to embodiments of the present disclosure, includes: administering a composition including any of the above-described ASO sequences, ASO variants, or chemical derivatives thereof to the target. The ASO may be a sequence of SEQ ID NOs: 29 to 86, as described above. The target may be in vivo or in vitro.

In some embodiments, the administering the ASO composition may include administering a composition comprising the ASO conjugated to a carrier peptide. In some embodiments, the administering of the ASO composition (whether alone or conjugated to a carrier peptide) may occur in a mouse or human subject (e.g., a patient with Huntington's disease).

In some embodiments, the target may be a cell selected from the group consisting of neurons, neuronal cells, brain cells, glial cells, astrocytes, neuronal supporting cells, and cells of the central nervous system (CNS). In some embodiments, the target (e.g., cell) includes or expresses a receptor that is targeted by the carrier peptide, for example, a nicotinic acetylcholine receptor. In some embodiments, the target may be within or across the BBB. As such, according to embodiments of the present disclosure, the administering the ASO composition to the target includes delivery of the ASO across the BBB. Any suitable method or manner of delivering the ASO across the BBB may be used without limitation. In some embodiments, delivery across the BBB may be accomplished by conjugating the ASO to one of the carrier peptides disclosed herein.

The relative amounts of effector agent (e.g., ASO) and carrier peptide in the ASO composition may be selected to maximize or increase the amount of ASO that is conjugated to carrier peptide and therefore available for delivery to the target. The concentration of the conjugate is related to the equilibrium constant for association (Kₐ) between the two molecules. Accordingly, the relative concentrations of the ASO and the carrier peptide may be modified or selected to account for various intermolecular forces (including electrostatic attraction) affecting the Kₐ. In some embodiments, a molar ratio of the effector agent to the carrier peptide may be 1:30 to 1:1, 1:25 to 1:1, 1:22 to 1:1, 1:20 to 1:1, 1:18 to 1:1, 1:15 to 1:1, 1:12 to 1:1, 1:10 to 1:1, 1:8 to 1:1, 1:6 to 1:1, 1:4 to 1:1, or 1:2 to 1:1.

In some embodiments, however, the molar ratio of the ASO and the carrier peptide may be 1:5 to 1:30, for example, 1:7 to 1:25; 1:7 to 1:22, 1:10 to 1:20, or 1:14 to 1:18. In some embodiments, the ratio of the ASO and the carrier peptide may be 1:15 or 1:19. And in some example embodiments, the ratio of the effector agent (e.g., anti-sense oligonucleotide) and the carrier peptide may be 1: greater than 10, for example, 1: greater than 10 to 1:30, 1: greater than 10 to 1:25, 1: greater than 10 to 1:22, 1: greater than 10 to 1:20, 1: greater than 10 to 1:18, 1: greater than 10 to 1:15. And in some alternative embodiments, the ratio of the effector agent and the carrier peptide may be 1: lower than 10, for example, 1:4 to 1: lower than 10, 1:2 to 1: lower than 10, or 1:7 to 1: lower than 10.

Additionally, in some embodiments, the ratio of the effector agent and the carrier peptide may be greater than 1:1. For example, in some embodiments, the ratio of the effector agent and the carrier peptide may be about 1:30 to 5:1, 1:25 to 5:1, 1:22 to 5:1, 1:20 to 5:1, 1:18 to 5:1, 1:15 to 5:1, 1:12 to 5:1, 1:10 to 5:1, 1:8 to 5:1, 1:6 to 5:1, 1:4 to 5:1, 1:2 to 5:1, 1:30 to 3:1, 1:25 to 3:1, 1:22 to 3:1, 1:20 to 3:1, 1:18 to 3:1, 1:15 to 3:1, 1:12 to 3:1, 1:10 to 3:1, 1:8 to 3:1, 1:6 to 3:1, 1:4 to 3:1, 1:2 to 3:1, 1:30 to 2:1, 1:25 to 2:1, 1:22 to 2:1, 1:20 to 2:1, 1:18 to 2:1, 1:15 to 2:1, 1:12 to 2:1, 1:10 to 2:1, 1:8 to 2:1, 1:6 to 2:1, 1:4 to 2:1, 1:2 to 2:1, 1:30 to 1.5:1, 1:25 to 1.5:1, 1:22 to 1.5:1, 1:20 to 1.5:1, 1:18 to 1.5:1, 1:15 to 1.5:1, 1:12 to 1.5:1, 1:10 to 1.5:1, 1:8 to 1.5:1, 1:6 to 1.5:1, 1:4 to 1.5:1, or 1:2 to 1.5:1.

In some embodiments of the method, the composition is a pharmaceutical composition. When the composition is prepared as a pharmaceutical solution, the ASO and the carrier peptide may be dissolved or suspended in any suitable aqueous carrier or solution that is acceptable for parenteral administration (such as a saline and/or dextrose solution). In some embodiments, for example, the solution may be a 0.5 wt % to 5 wt % saline solution (e.g., a 0.5 wt % to 3 wt %, or 0.9 wt % to 2 wt % saline solution) or a 1 wt % to 10 wt % dextrose solution (e.g., a 2 wt % to 8 wt %, or 3 wt % to 5 wt % dextrose solution). In some embodiments, the solution may include additional stabilizing agents in the art, such as PEG8000 (e.g., at a concentration of 0.01 wt % to 1 wt %, for example, 0.05 wt % to 0.5 wt %, 0.1 wt % to 0.4 wt %, or 0.2 wt % to 0.3 wt %) and/or Polysorbate 80 (e.g., at a concentration of 0.01 wt % to 1.5 wt %, for example, 0.05 wt % to 1.2 wt %, 0.1 wt % to 1 wt, 0.15 wt % to 0.8 wt %, 0.2 wt % to 0.7 wt %, or 0.3 wt % to 0.6 wt %).

In some embodiments, the ASO may be included in the pharmaceutical solution in an amount of 0.005% to 1% by weight, for example, 0.015% to 0.5%, 0.03% to 0.25%, 0.04% to 0.1%, or 0.05% to 0.08% by weight. For example, a pharmaceutical solution may include about 2.5 to 5.0 μg of effector agent (about 15 to 30 μg of conjugate) in 100 μL of solution. For example, in some embodiments, the pharmaceutical solution may include about 5.0 μg of the effector agent (about 30 μg of conjugate) in 100 μL of solution. And in some embodiments, for example, the pharmaceutical solution may include about 2.5 μg of the effector agent (about 15 μg of conjugate) in 100 μL of solution.

As discussed above, in some embodiments, the method may further include conjugating the ASO to the carrier peptide, prior to administering the composition. The chemical or biochemical steps used to achieve such a conjugation depend on the particular linker or interaction between the two molecules, and those having ordinary skill in the art are capable of selecting suitable methods, parameters, etc., to achieve the conjugation.

Administration of the composition to the target may result in an observable decrease in HTT mRNA levels and/or HTT protein expression. The decrease in HTT mRNA levels in targeted cells and tissues (e.g., particular regions of the brain, including those that are more strongly affected by HD) can be measured by extracting RNA from a suitable sample of the target (e.g., from cells including a receptor that can be targeted by the carrier peptide, such as a nACHR receptor), generating cDNA from a set or predetermined volume of the RNA extract, and subjecting the cDNA to PCR. The relative decrease (e.g., knock-down) of HTT mRNA with respect to an untreated control can be quantified by comparing the change in HTT mRNA levels in the presence and absence of the ASO:carrier peptide composition with an unrelated mRNA that is expected to remain at a constant level even in the presence of the ASO:carrier peptide composition. For example, the unrelated mRNA standard may be the synuclein alpha (SNCA) protein, which is also expressed in neuronal cells.

In some embodiments, the method of treating HD according to embodiments of the present disclosure may result in a statistically significant decrease in (e.g., knock-down of) HTT mRNA levels (e.g., with a P-value of less than 0.05, and in some embodiments less than 0.01). For example, the methods according to embodiments of the present disclosure may result in a decrease in HTT mRNA levels of greater than 20%, for example, a decrease of 20% to 90%, 30% to 90%, 40% to 85%, 50% to 80%, or 60% to 75%.

In some embodiments, for example, when the target is an in vitro neuronal cell culture, HTT mRNA levels may be decreased by 25% to 90%, for example, 35% to 85%, 45% to 75%, or 55% to 70% relative to untreated (control) levels.

In some embodiments, for example, when the target is in vivo in a mouse (e.g., wild-type mouse), HTT mRNA levels may be decreased by 40% to 65% in the hippocampus, for example, 45% to 60%, or 50% to 55%, and by 15% to 40% in the striatum, for example, 20% to 35% or 25% to 30% relative to untreated (control) levels.

The following Examples are presented for illustrative purposes only, and do not limit the scope or content of the present application.

### Example 1. Selection of Lead ASOs

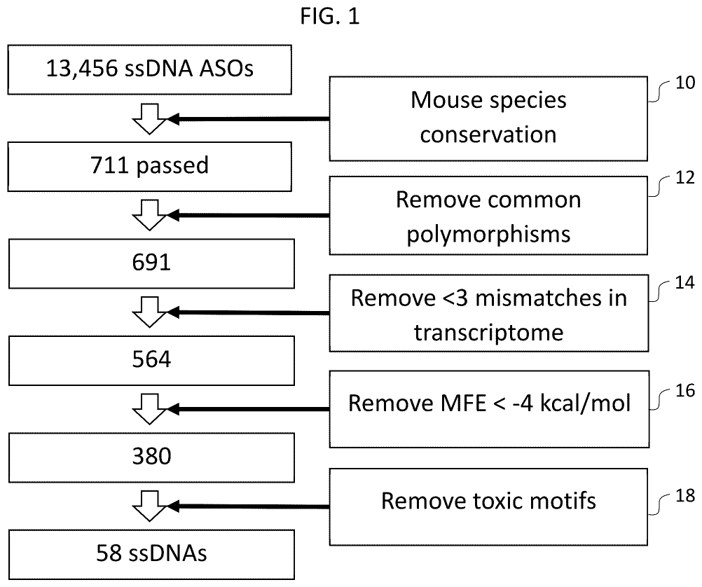

ASO sequences with the potential to bind to HTT mRNA and block its translation to protein were identified using in silico methods as follows. FIG. 1 is a flowchart diagram summarizing the selection procedure. As shown in box 10 of the flowchart, 13,456 complementary 20-mer oligonucleotides to human HTT mRNA were identified, and then filtered to include only the 711 sequences that are conserved between humans and mice.

As shown in boxes 12 and 14, potentially risky candidates were eliminated, including 20 ASOs matched to mRNA sequences having nucleotide polymorphisms with an occurrence frequency of greater than 1%, and 127 ASOs having 0, 1 or 2 mismatches (e.g., 90% to 100% complementarity) to the mRNAs of human genes other than HTT.

As shown in box 16, the minimal free energy (MFE) of each oligonucleotide was calculated. Lower MFEs are correlated with higher chances of forming a strong secondary structure (e.g., a hairpin loop, a pseudoknot, etc.), which is undesirable in an ASO. Oligomers predicted to form secondary structures with a MFE less than −4 kcal/mol were eliminated, resulting in 380 remaining candidates.

As shown in box 18, candidates with known toxic motifs (such as tgc, tcc, and gc) were eliminated, resulting in 58 remaining ASO candidates (SEQ ID NOS:29 to 86). Such toxic motifs are further described in e.g., Burdick et al., “Sequence motifs associated with hepatotoxicity of locked nucleic acid-modified antisense oligonucleotides,” Nucleic Acids Res., 2014, 42(8), pg. 4882-4891, the entire content of which is incorporated herein by reference.

The ASO candidates of (SEQ ID NOS:29 to 86) had a total guanine and cytosine content of between 20% to 60%. Five ASO candidates (ASO1 to ASO5 having SEQ ID NOs: 87, 97, 99, 100 and 125), each having a total guanine and cytosine content of 35% to 45%, were selected for further study.

### Example 2. In Vitro Knockdown of HTT mRNA by Selected ASOs

The selected HTT ASO candidates were initially evaluated for HTT knockdown efficacy and specificity in vitro, using lynx1-loop2-derived carrier peptides to mediate delivery into human neuronal cells.

The key genes involved in lynx1-loop2-mediated delivery via the nACHRs include the nicotinic receptor alpha7 subunit (CHRNA7), nicotinic receptor alpha4 subunit (CHRNA4), nicotinic receptor beta2 subunit (CHRNB2), and NACHO. Because differentiated SHSY5Y human cells express all such genes of interest, they were selected to test for knockdown of HTT mRNA. The HTT mRNA knockdown efficiency was assessed by comparison to synuclein alpha (SNCA), which is a neuronally expressed gene that is regulated independently of HTT.

Methods: SHSY5Y cells were grown according to standard protocols as specified by the vendor (ATCC). When cells reached 80% confluency, the cells were passaged and 160K cells were plated in 0.5 mL of growth media in a 24-well plate. 24 hours later, the growth media was replaced with differentiation media (day 1 of differentiation protocol). Two days later, 30% of the differentiation media was replaced with fresh differentiation media (day 3 of differentiation protocol). ASO1 to ASO5 (SEQ ID NOs: 87, 97, 99, 100 and 125) were tested with Peptide 1 (SEQ ID NO:16) or Peptide 4 (SEQ ID NO:17) as follows.

On day 4 of the differentiation protocol, Peptide 1 (SEQ ID NO:16):ASO or Peptide 4 (SEQ ID NO:17):ASO complexes were mixed in DMEMF12 media and incubated for 15 min at room temperature (e.g., 25 °C). At the end of the incubation period, 0.1 mL of the differentiation media was replaced with 0.1 mL of the peptide:ASO complexes. The final concentrations in the cell growth media were 15 μM peptide:1 μM ASO (15:1 ratio).

24 hours after exposure to the complexes, the cells were washed once with PBS, and RNA was extracted using an RNeasy Mini Kit (Qiagen, #74106) with an on column DNase I treatment (Qiagen, RNase-Free DNase Set, #79256). cDNA was generated from 0.5 μg of RNA using the KIT CDNA SUPERMIX QSCRIPT (Quanta, VWR, #101414-108). Each qPCR reaction contained 20 ng of cDNA. The KIT QPCR FASTMIX II ROX (Quanta, VWR, #97065-998) was used for the qPCR reaction, and triplicates were run on the Applied Biosystems™ 7500 Real-Time PCR System. Knockdown of appropriate HTT target mRNA was assessed by comparison to SNCA using suitable primers for RT-qPCR: Taqman assay Hs00918174_m1 (HTT1) for HTT; and Taqman assay Hs00240907_m1 for alpha-synuclein (SNCA). The qPCR values of the HTT and SNCA gene were normalized to glyceraldehyde 3-phosphate dehydrogenase (GAPDH, Taqman assay Hs02786624_g1) and peptidylprolyl isomerase (PPIA, Taqman assay Hs04194521_s).

Result Differentiated SHSY5Y cells showed a statistically significant decrease of HTT mRNA expression upon exposure to ASO3 (SEQ ID NO:99) when complexed with either of Peptide 1 (SEQ ID NO:16) or Peptide 4 (SEQ ID NO:17). No significant change in expression of SNCA was observed in those same samples.

The cells showed a decrease in HTT mRNA expression upon exposure to ASO1, ASO2, ASO3, ASO4, and ASO5 (SEQ ID NOS: 87, 97, 99, 100, and 125) when complexed with either of Peptide 1 (SEQ ID NO:16) or Peptide 4 (SEQ ID NO:17), but also showed a simultaneous decrease in SNCA mRNA expression, indicating that these ASOs had comparatively lower specificity for HTT.

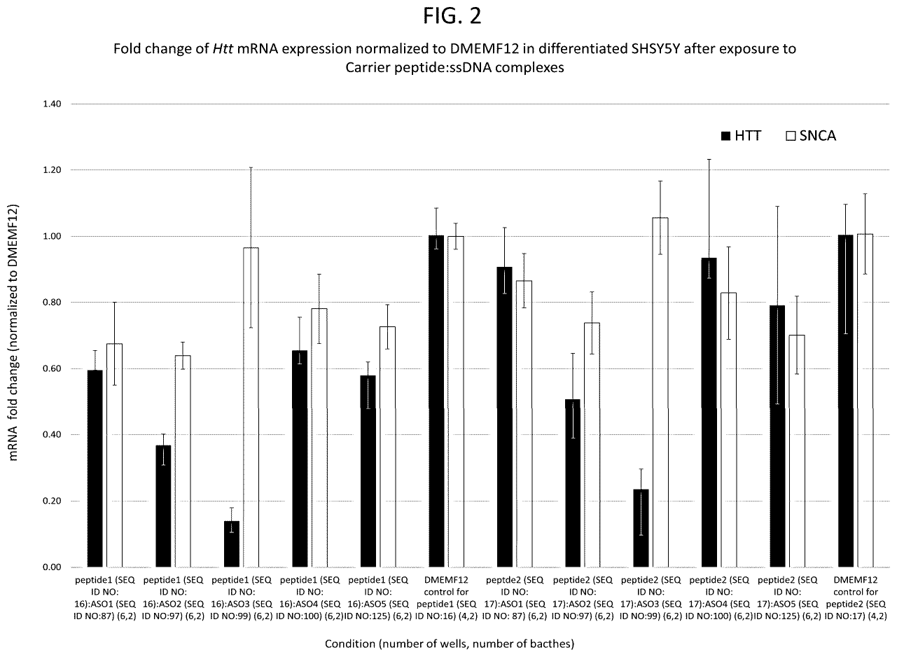

Table 4 summarizes the change in measured mRNA levels as normalized against the change in HTT1 mRNA in the DMEMF12-only control; values in bold highlight statistically significant knockdowns. FIG. 2 presents the same data in bar chart form; the error bars represent the standard deviation. In particular, SHSY5Y exposure to Peptide 1 (SEQ ID NO:16):ASO3 (SEQ ID NO:99) was correlated with an 86% (P<0.01) reduction of HTT target mRNA. Exposure to Peptide 4 (SEQ ID NO:17):ASO3 (SEQ ID NO:99) was correlated with a 76% (P<0.01) reduction of HTT target mRNA. Exposure to DMEMF12 (e.g., in the absence of any ASO or peptide) did not show any statistically significant effect on HTT mRNA levels.

TABLE 4

| | | | Fold Change | | | |
|---|---|---|---|---|---|---|
| Peptide:ASO | Fold Change | | Standard | | | |
| (number of wells, number of | Average | | Deviation | | p value | |
| batches) | HTT | SNCA | HTT | SNCA | HTT | SNCA |
| Peptide 1 (SEQ ID NO: 16):ASO1 | 0.60 | 0.68 | 0.06 | 0.13 | 0.00 | 0.00 |
| (SEQ ID NO: 87) | | | | | | |
| 15 uM-1 uM (6, 2) | | | | | | |
| Peptide 1 (SEQ ID NO: 16):ASO2 | 0.37 | 0.64 | 0.03 | 0.04 | 0.00 | 0.00 |
| (SEQ ID NO: 97) | | | | | | |
| 15 uM-1 uM (6, 2) | | | | | | |
| Peptide 1 (SEQ ID NO: 16):ASO3 | 0.14 | 0.97 | 0.04 | 0.24 | 0.00 | 0.80 |
| (SEQ ID NO: 99) | | | | | | |
| 15 uM-1 uM (6, 2) | | | | | | |
| Peptide 1 (SEQ ID NO: 16):ASO4 | 0.65 | 0.78 | 0.10 | 0.10 | 0.00 | 0.01 |
| (SEQ ID NO: 100) | | | | | | |
| 15 uM-1 uM (6, 2) | | | | | | |
| Peptide 1 (SEQ ID NO: 16):ASO5 | 0.58 | 0.73 | 0.04 | 0.07 | 0.00 | 0.00 |
| (SEQ ID NO: 125) | | | | | | |
| 15 uM-1 uM (6, 2) | | | | | | |
| DMEMF12 control | 1.00 | 1.00 | 0.08 | 0.04 | 1.00 | 1.00 |
| for Peptide 1 (4, 2) | | | | | | |
| Peptide 4 (SEQ ID NO: 17):ASO1 | 0.91 | 0.87 | 0.12 | 0.08 | 0.31 | 0.09 |
| (SEQ ID NO: 87) | | | | | | |
| 15 uM-1 uM (6, 2) | | | | | | |
| Peptide 4 (SEQ ID NO: 17):ASO2 | 0.51 | 0.74 | 0.14 | 0.09 | 0.00 | 0.01 |
| (SEQ ID NO: 97) | | | | | | |
| 15 uM-1 uM (6, 2) | | | | | | |
| Peptide 4 (SEQ ID NO: 17):ASO3 | 0.24 | 1.06 | 0.06 | 0.11 | 0.00 | 0.57 |
| (SEQ ID NO: 99) | | | | | | |
| 15 uM-1 uM (6, 2) | | | | | | |
| Peptide 4 (SEQ ID NO: 17):ASO4 | 0.94 | 0.83 | 0.30 | 0.14 | 0.70 | 0.10 |
| (SEQ ID NO: 100) | | | | | | |
| 15 uM-1 uM (6, 2) | | | | | | |
| Peptide 4 (SEQ ID NO: 17):ASO5 | 0.79 | 0.70 | 0.30 | 0.12 | 0.25 | 0.01 |
| (SEQ ID NO: 125) | | | | | | |
| 15 uM-1 uM (6, 2) | | | | | | |
| DMEMF12 control | 1.00 | 1.01 | 0.09 | 0.12 | 1.00 | 1.00 |
| for Peptide 4 (4, 2) | | | | | | |

In Table 4, “Peptide 1” corresponds to SEQ ID NO. 16; Peptide 4” corresponds to SEQ ID NO. 17; and “ASO1” to “ASO5” correspond to SEQ ID NOS. 87, 97, 99, 100 and 125, respectively.

Conclusions: Statistically significant and specific knockdown of HTT mRNA by ASO3 (SEQ ID NO. 99), when complexed with either Peptide 1 (SEQ ID NO:16) or Peptide 4 (SEQ ID NO:17), was observed in vitro in differentiated SHSY5Y cell lines. Based on the data from this study, ASO3 was further tested in vivo for their efficacy in specific brain regions, discussed below.

To assess differences in knockdown according to differences in concentration of the ASO, selected ASOs were evaluated for HTT knockdown efficacy and specificity in vitro, using different ratios of the lynx1-loop2-derived carrier peptides to the ASOs. The protocol was generally as described above, except that the final concentrations of the peptide:ASO mixtures in the cell growth media were varied between a 15:1 ratio (15 μM peptide:1 μM ASO), a 10:1 ratio (10 μM peptide:1 μM ASO), and a 4:1 ratio (4 μM peptide:1 μM ASO). Also, the final peptide:ASO mixtures (having different peptide:ASO ratios) were prepared in a solution of dextrose with 0.6% polysorbate 80. And knockdown of appropriate HTT target mRNA was assessed by comparison to SNCA using the HTT1 and SNCA primers noted above in addition to another primer: Taqman assay Hs00918178_m1 (HTT3) for HTT.

Result Differentiated SHSY5Y cells showed a statistically significant decrease of HTT mRNA expression upon exposure to all tested ASOs (including those with ASO3 (SEQ ID NO:99), ASO6 (SEQ ID NO:41) and ASO2 (SEQ ID NO:97)) when conjugated to Peptide 1 (SEQ ID NO:16).

The cells showed a decrease in HTT mRNA expression upon exposure to ASO3, ASO6 and ASO2 (SEQ ID NOS: 99, 41 and 91, respectively) when conjugated to Peptide 1 (SEQ ID NO:16).

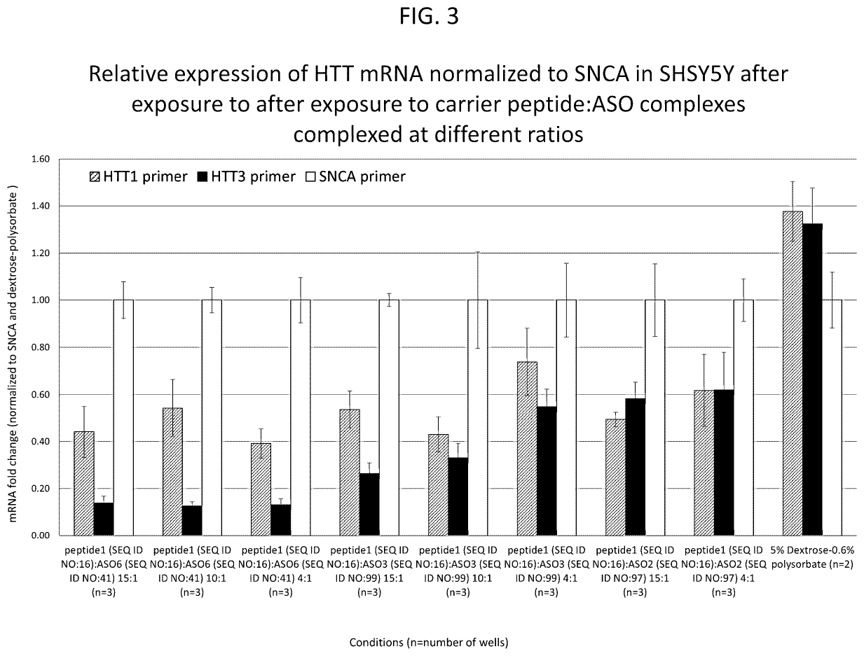

Table 5 summarizes the change in measured mRNA levels normalized to SNCA; values in bold highlight statistically significant knockdowns. FIG. 3 presents the same data in bar chart form; the error bars represent the standard deviation. As shown in Table 5 and FIG. 3, all of the peptide:ASO conjugates at all ratios exhibited significant knockdown, with the ASO6 (SEQ ID NO:41) conjugates showing better knockdown than the ASO3 (SEQ ID NO:99) and ASO2 (SEQ ID NO:97) conjugates. And the ASO6 (SEQ ID NO:41) conjugates exhibited similar knockdown at all tested ratios (i.e., 15:1, 10:1 and 4:1). But while all ASO3 (SEQ ID NO:99) conjugates exhibited significant knockdown, the 15:1 and 10:1 ratios exhibited better knockdown than the 4:1 ratio. Similarly, while all ASO2 (SEQ ID NO:9.) conjugates exhibited significant knockdown, the 15:1 ratio exhibited better knockdown than the 4:1 ratio. Exposure to Dextrose-0.6% alone (e.g., in the absence of any ASO or peptide) did not show any statistically significant effect on HTT mRNA levels.

TABLE 5

| | | | | Fold Change Standard | | | | | |
|---|---|---|---|---|---|---|---|---|---|
| Peptide:ASO | Fold Change Average | | | Deviation | | | Normalized p value | | |
| (n = number of wells) | HTT1 | HTT3 | SNCA | HTT1 | HTT3 | SNCA | HTT1 | HTT3 | SNCA |
| 15:1 Peptide 1 (SEQ ID | 0.44 | 0.14 | 1.00 | 0.11 | 0.03 | 0.08 | 0.01 | 0.00 | 1.00 |
| NO: 16):ASO6 (SEQ ID | | | | | | | | | |
| NO: 41) | | | | | | | | | |
| (n = 3) | | | | | | | | | |
| 10:1 Peptide 1 (SEQ ID | 0.54 | 0.13 | 1.00 | 0.12 | 0.02 | 0.05 | 0.01 | 0.00 | 1.00 |
| NO: 16):ASO6 (SEQ ID | | | | | | | | | |
| NO: 41) | | | | | | | | | |
| (n = 3) | | | | | | | | | |
| 4:1 Peptide 1 (SEQ ID | 0.39 | 0.13 | 1.00 | 0.06 | 0.03 | 0.10 | 0.00 | 0.00 | 1.00 |
| NO: 16):ASO6 (SEQ ID | | | | | | | | | |
| NO: 41) | | | | | | | | | |
| (n = 3) | | | | | | | | | |
| 15:1 Peptide 1 (SEQ ID | 0.54 | 0.26 | 1.00 | 0.08 | 0.04 | 0.03 | 0.01 | 0.00 | 1.00 |
| NO: 16):ASO3 (SEQ ID | | | | | | | | | |
| NO: 99) | | | | | | | | | |
| (n = 3) | | | | | | | | | |
| 10:1 Peptide 1 (SEQ ID | 0.43 | 0.33 | 1.00 | 0.07 | 0.06 | 0.20 | 0.00 | 0.00 | 1.00 |
| NO: 16):ASO3 (SEQ ID | | | | | | | | | |
| NO: 99) | | | | | | | | | |
| (n = 3) | | | | | | | | | |
| 4:1 Peptide 1 (SEQ ID | 0.74 | 0.55 | 1.00 | 0.14 | 0.07 | 0.16 | 0.03 | 0.01 | 1.00 |
| NO: 16):ASO3 (SEQ ID | | | | | | | | | |
| NO: 99) | | | | | | | | | |
| (n = 3) | | | | | | | | | |
| 15:1 Peptide 1 (SEQ ID | 0.49 | 0.58 | 1.00 | 0.03 | 0.07 | 0.15 | 0.02 | 0.05 | 1.00 |
| NO: 16):ASO2 (SEQ ID | | | | | | | | | |
| NO: 97) | | | | | | | | | |
| (n = 3) | | | | | | | | | |
| 4:1 Peptide 1 (SEQ ID | 0.62 | 0.62 | 1.00 | 0.15 | 0.16 | 0.09 | 0.02 | 0.03 | 1.00 |
| NO: 16):ASO2 (SEQ ID | | | | | | | | | |
| NO: 97) | | | | | | | | | |
| (n = 3) | | | | | | | | | |
| Dextrose-0.6% polysorbate | 1.38 | 1.32 | 1.00 | 0.13 | 0.15 | 0.12 | — | — | — |
| 80 only (1X) | | | | | | | | | |
| (n = 2) | | | | | | | | | |

### Example 3. In Vivo Knock-Down of Mouse HTT mRNA by ASO03 (SEQ ID NO:99) with Peptide 4 (SEQ ID NO:17)

Methods: Animals were allowed to acclimate at an approved vendor (Charles River Laboratories International, Inc., (CRL), Shrewsbury, MA) for at least 2 days prior to the start of the experiment. Male wild-type C57bl6 mice were 2-5 months of age, fed Teklad global rodent chow, and were housed in groups of 2 to 5 per cage. ASOs were synthesized by Integrated DNA Technologies (IDT, Coralville, IA). Carrier peptides were synthesized by Lifetein (Hillsborough, NJ).

ASO3 (SEQ ID NO: 99) was administrated to each animal using the following procedure. Briefly, 25 μg of ASO3 (SEQ ID NO:99) and Peptide 4 (SEQ ID NO:17) peptide were mixed in vital dye (Evans Blue)/saline solution to produce a 15:1 or 19:1 molar ratio of Peptide 4 to ASO3, and incubated for 15 minutes at room temperature. The Peptide 4:ASO3 complexes were then delivered as a 100 μL injection per mouse via the lateral tail vein. An identical volume of saline solution was injected in control animals.

Knockdown of HTT mRNA in the hippocampus (HP) and striatum (ST) was assessed by RT-qPCR. Seventy-two hours post-dose, animals were sacrificed and whole brains were removed without perfusion. Left-side brain tissues (HP and ST) were stored in RNAlater solution prior to RNA extraction. RNA was extracted from the HP and ST brain regions using an RNeasy Mini Kit (Qiagen, #74106) with an on column DNAse I treatment (Qiagen, #79256). cDNA was generated from 1 μg of RNA using the KIT CDNA SUPERMIX QSCRIPT (Quanta, VWR, #101414-108). Each qPCR reaction contained 30-50 ng of cDNA. The KIT QPCR FASTMIX II ROX (Quanta, VWR, #97065-998) was used for the qPCR reaction; triplicates were run on the Applied Biosystems™ 7500 Real-Time PCR System. Knockdown of HTT target mRNA was assessed by comparison to SNCA using suitable primers for RT-qPCR: Taqman assay Mm01213763 for HTT, and Taqman assay Mm01188700_m1 for SNCA. The qPCR values of the HTT and SNCA gene were normalized to glyceraldehyde 3-phosphate dehydrogenase (GAP DH, Taqman assay Mm99999915_g1) and peptidylprolyl isomerase (PPIA, Taqman assay Mm02342430_g1).

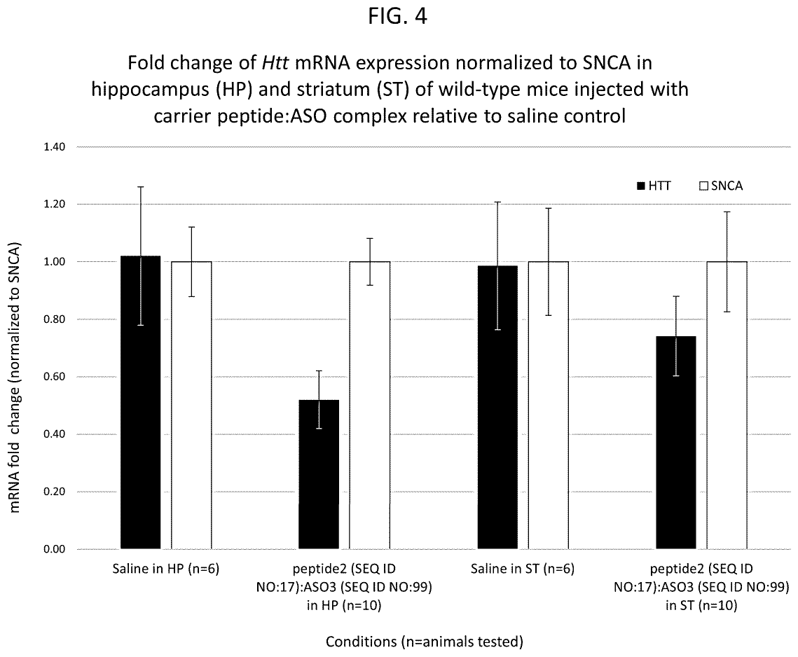

Result: ASO3 (SEQ ID NO:99) complexed with Peptide 4 (SEQ ID NO:17) showed a statistically significant decrease in HTT mRNA expression in the HP and ST of wild-type mice. Table 6 summarizes the change in HTT mRNA levels as normalized against SNCA and relative to the saline control; values in bold highlight statistically significant knockdowns. FIG. 4 presents the same data in bar chart form; the error bars represent the standard deviation. In particular, a 48% (P<0.01) reduction of HTT mRNA was observed in the HP of mice injected with Peptide 4 (SEQ ID NO:17):ASO3 (SEQ ID NO:99), and a 26% (P<0.01) reduction was observed in the ST.

TABLE 6

| | | | Standard | | | |
|---|---|---|---|---|---|---|
| | Fold Change | | Deviation | | | |
| HTT mRNA knockdown in wild-type | Average | | Average | | p value | |
| mice HP and ST | HTT | SNCA | HTT | SNCA | HTT | SNCA |
| Saline in HP (n = 6) | 1.02 | 1.00 | 0.24 | 0.12 | 1.00 | 1.00 |
| Peptide 4 (SEQ ID NO: 17):ASO3 | 0.52 | 1.00 | 0.10 | 0.08 | 0.00 | 1.00 |
| (SEQ ID NO: 99) in HP (n = 10) | | | | | | |
| Saline in ST (n = 6) | 0.99 | 1.00 | 0.22 | 0.19 | 0.00 | 1.00 |
| Peptide 4 (SEQ ID NO: 17):ASO3 | 0.74 | 1.00 | 0.14 | 0.17 | 1.00 | 1.00 |
| (SEQ ID NO: 99) in ST (n = 10) | | | | | | |

In Table 6, “Peptide 4” corresponds to SEQ ID NO. 17; and “ASO3” corresponds to SEQ ID NO: 99.

Conclusions: Statistically significant and specific knockdown of HTT mRNA was observed in vivo in wild-type mice treated with ASO3 (SEQ ID NO: 99) complexed with Peptide 4 (SEQ ID NO:17).

### Example 4. Chronic Dosage of 25 μg in Humanized zQ175 Huntington's Mice

Methods: Male and female zQ175 mice at PsychoGenics (Paramus, New Jersey) were acclimated for several days before the start of the experiment. Fasting was not required for this study. The ASOs and carrier peptides were obtained as in Example 3.

Three groups of 7-8 mice each were tested in this study, including: 1) ASO6 conjugate treated mice; 2) vehicle control mice; and 3) Wild-Type (WT) mice. Each mouse was 12 weeks old at the initiation of the therapy, and was administered one dose every two weeks for a total of 6 months. Each dose included an ASO6 (SEQ ID NO:41) conjugate administrated to each animal using the following procedure. Briefly, 25 μg of ASO6 (SEQ ID NO:41) and Peptide 1 (SEQ ID NO:16) were mixed in a dextrose-0.6% polysorbate 80 solution to produce a 10:1 molar ratio of peptide to ASO, and incubated for 15 minutes at room temperature. The peptide:ASO conjugates were then delivered with a dose of 25 μg of ASO6 (SEQ ID NO:41) as a 100 μL injection per mouse via the lateral tail vein. An identical volume of a dextrose-0.6% polysorbate 80 solution was injected in vehicle control animals.

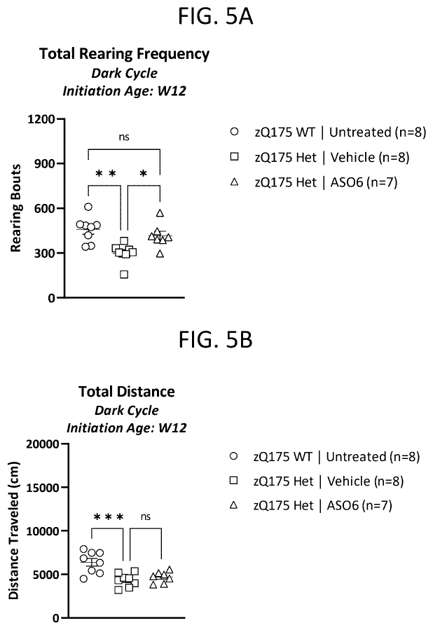

Total rearing frequency, total distance traveled, and post-treatment mutant HTT protein levels in the cortex (CTX) and striatum (ST) were assessed. Soluble expanded mutant HTT levels in both the CTX and the ST were assessed via Assay 6 (2B7/MW1-ST), and aggregated mutant HTT levels in both the CTX and ST were assessed via Assay 45 (MW8/4C9-ST). The results of these assessments are shown in FIG. 5A (total rearing frequency), 5B (total distance traveled), 6A (soluble expanded mutant HTT levels in the CTX), 6B (aggregated mutant HTT levels in the CTX), 6C (soluble expanded mutant HTT levels in the ST) and 6D (aggregated mutant HTT levels in the ST). Tables 7 and 8, below, show the total values (and standard deviations (SD)) for the distance traveled and rearing frequency. Tables 9-12, below, show the values for the soluble expanded and aggregated mHTT levels (average±standard deviation (SD)) in the CTX (Tables 9 and 10) and ST (Tables 11 and 12).

TABLE 7
Distance Traveled (25 μg study)

| Group | Total Distance in cm (average ± SD) |
|---|---|
| Vehicle control | 4334.93 ± 760.80 |
| ASO6 | 4676.93 ± 637.12 |
| WT | 6380.29 ± 1251.61 |

TABLE 8
Rearing Frequency (25 μg study)

| Group | Total Rearing Bouts (average ± SD) |
|---|---|
| Vehicle control | 298.50 ± 64.25 |
| ASO6 | 415.86 ± 82.07 |
| WT | 457.00 ± 87.10 |

TABLE 9
Expanded mHTT in CTX in 25 μg study (Assay 6: 2B7/MW1-ST)

| | Expanded mHTT | % Expanded | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 648.73 ± 32.64 | 100 | — |
| ASO6 | 572.90 ± 50.34 | 88.31 | 11.69 |

TABLE 10
Aggregated mHTT in CTX in 25 μg study
(Assay 45: MW8/4C9-ST)

| | Aggregated mHTT | % Aggregated | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 1407.76 ± 107.83 | 100 | — |
| ASO6 | 1331.59 ± 179.63 | 94.59 | 5.41 |

TABLE 11
Soluble expanded mHTT in ST in 25
μg study (Assay 6: 2B7/MW1-ST)

| | Expanded mHTT | % Expanded | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 597.07 ± 60.77 | 100 | — |
| ASO6 | 679.89 ± 119.06 | 113.89 | −13.9 |

TABLE 12
Aggregated mHTT in ST in 25 μg study (Assay 45: MW8/4C9-ST)

| | Aggregated mHTT | % Aggregated | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 1962.37 ± 240.22 | 100 | — |
| ASO6 | 1714.72 ± 321.00 | 87.38 | 12.6 |

Results: As shown in FIG. 5A, the Total Rearing Frequency in the ASO6 treated group was similar to that of the WT group (reversal of phenotype). There were significant differences in total rearing between the treatment groups (F (3, 21)=8.191, p=0.0003) with the untreated WT group (p<0.01) and the group treated with ASO6 (p<0.05) rearing significantly more compared to the group treated with the vehicle control. It is important to note that the ASO6 treated group is statistically different than the vehicle group, and the ASO6 group is statistically the same as the wild-type group, suggesting the ASO6 treatment prevents neurodegeneration that leads to reduced behavior.

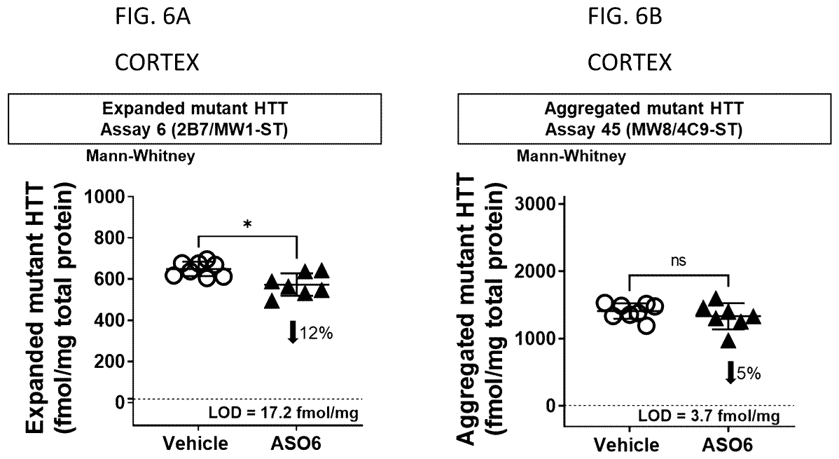

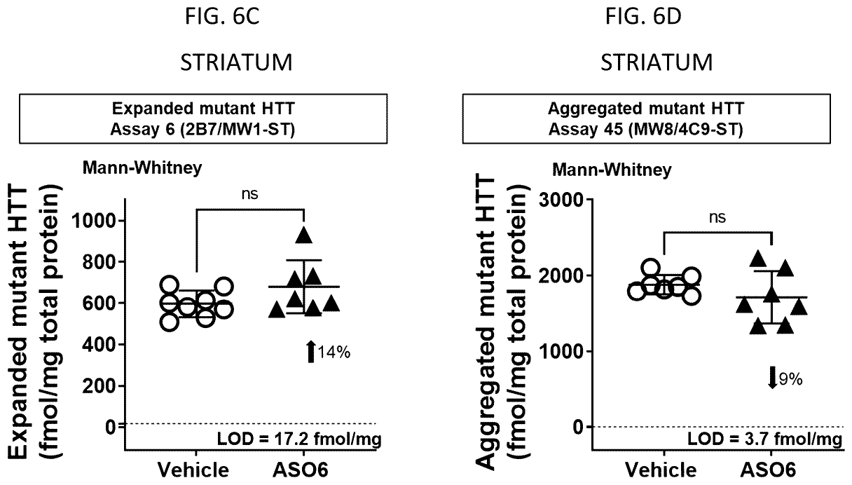

This improvement in function correlates well with the reduction of the soluble protein level in the cortex (12%) and no change in the aggregated protein in the cortex, soluble or aggregated protein in the striatum. See FIGS. 6A through 6D.

### Example 5. Chronic Dosage of 2.5 μg in Humanized zQ175 Huntington's Mice

Methods: Male and female zQ175 mice at PsychoGenics (Paramus, New Jersey) were acclimated for several days before the start of the experiment. Fasting was not required for this study. The ASOs and carrier peptides were obtained as in Example 3.

Three groups of 1-8 mice each were tested in this study, including: 1) ASO6 conjugate treated mice; 2) ASO4 conjugate treated mice; and 3) vehicle control mice. Each mouse was 15 weeks old at the initiation of the therapy, and was administered one dose every two weeks for a total of 3 months. Each dose included an ASO6 (SEQ ID NO:41) or ASO4 (SEQ ID NO:100) conjugate administrated to each animal using the following procedure. Briefly, 2.5 μg of ASO6 (SEQ ID NO:41) or ASO4 (SEQ ID NO:100) and Peptide 1 (SEQ ID NO:16) were mixed in a dextrose-0.6% polysorbate 80 solution to produce a 10:1 molar ratio of peptide to ASO, and incubated for 15 minutes at room temperature. The peptide:ASO conjugates were then delivered with a dose of 2.5 μg of ASO6 (SEQ ID NO:41) or ASO4 (SEQ ID NO:100) as a 100 μL injection per mouse via the lateral tail vein. An identical volume of a dextrose-0.6% polysorbate 80 solution was injected in vehicle control animals.

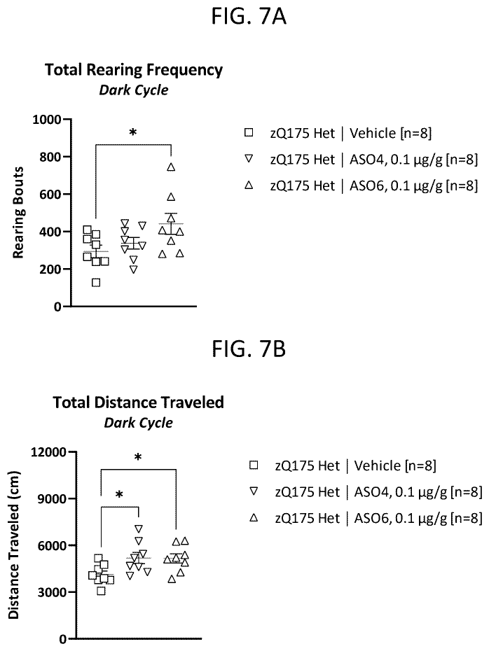

Total rearing frequency, total distance traveled, and post-treatment expanded and aggregated mutant HTT levels in the cortex (CTX) and striatum (ST) were assessed. Expanded mutant HTT levels in both the CTX and the ST were assessed via Assay 6 (2B7/MW1-ST), and aggregated mutant HTT levels in both the CTX and ST were assessed via Assay 45 (MW8/4C9-ST). The results of these assessments are shown in FIGS. 7A (total rearing frequency), 7B (total distance traveled), 8A (soluble expanded mutant HTT levels in the CTX), 8B (aggregated mutant HTT levels in the CTX), 8C (soluble expanded mutant HTT levels in the ST) and 8D (aggregated mutant HTT levels in the ST). Tables 13 and 14, below, show the total values (and standard deviations (SD)) for the distance traveled and rearing frequency. Tables 15-18, below, show the values for the expanded and aggregated mHTT levels (average±standard deviation (SD)) in the CTX (Tables 15 and 16) and ST (Tables 17 and 18).

TABLE 13
Distance Traveled (2.5 μg study)

| Group | Total Distance in cm (average ± SD) |
|---|---|
| Vehicle control | 4123.30 ± 658.37 |
| ASO6 | 5162.48 ± 854.98 |
| ASO4 | 5192.99 ± 1027.70 |

TABLE 14
Rearing Frequency (2.5 μg study)

| Group | Total Rearing Bouts (average ± SD) |
|---|---|
| Vehicle control | 294.75 ± 93.82 |
| ASO6 | 441.38 ± 158.53 |
| ASO4 | 337.88 ± 87.34 |

TABLE 15
Expanded mHTT in Cortex in 2.5
μg study (Assay 6: 2B7/MW1-ST)

| | Expanded mHTT | % Expanded | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 582.14 ± 53.96 | 100 | — |
| ASO6 | 481.25 ± 44.16 | 82.7 | 17.3 |
| ASO4 | 590.5 ± 34.12 | 101.4 | −1.4 |

TABLE 16
Aggregated mHTT in Cortex in 2.5
μg study (Assay 45: MW8/4C9-ST)

| | Aggregated mHTT | % Aggregated | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 1035.86 ± 78.36 | 100 | — |
| ASO6 | 871.13 ± 85.71 | 84.1 | 15.9 |
| ASO4 | 828.38 ± 100.61 | 80.0 | 20.0 |

TABLE 17
Soluble Expanded mHTT in Striatum
2.5 μg study (Assay 6: 2B7/MW1-ST)

| | Expanded mHTT | % Expanded | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 598.78 ± 56.24 | 100 | — |
| ASO6 | 514.63 ± 76.88 | 85.9 | 14.1 |
| ASO4 | 559.25 ± 55.48 | 93.4 | 6.6 |

TABLE 18
Aggregated mHTT in Striatum in 2.5
μg study (Assay 45: MW8/4C9-ST)

| | Aggregated mHTT | % Aggregated | % mHTT KD |
|---|---|---|---|
| | (fmol/mg total | mHTT based on | based on |
| | protein; average ± | 100% vehicle | 100% vehicle |
| Group | SD) | control | control |
| Vehicle control | 1568.63 ± 130.99 | 100 | — |
| ASO6 | 1570.88 ± 273.68 | 100.1 | −0.1 |
| ASO4 | 1575.63 ± 217.824 | 100.4 | −0.4 |

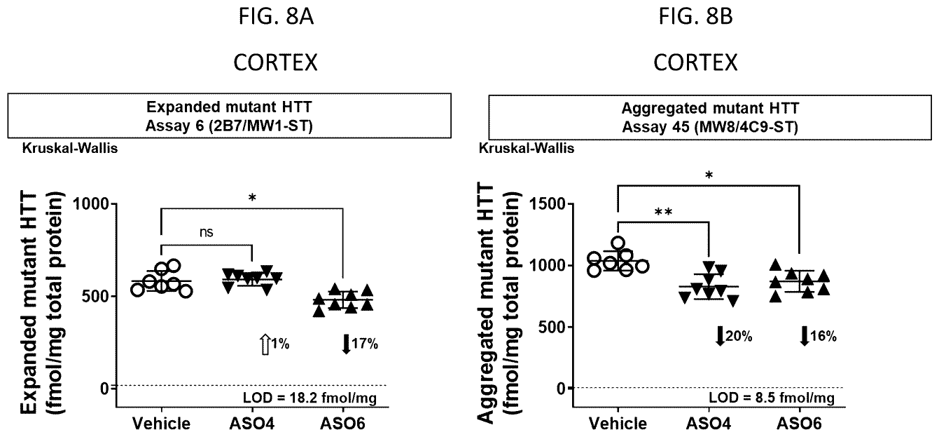

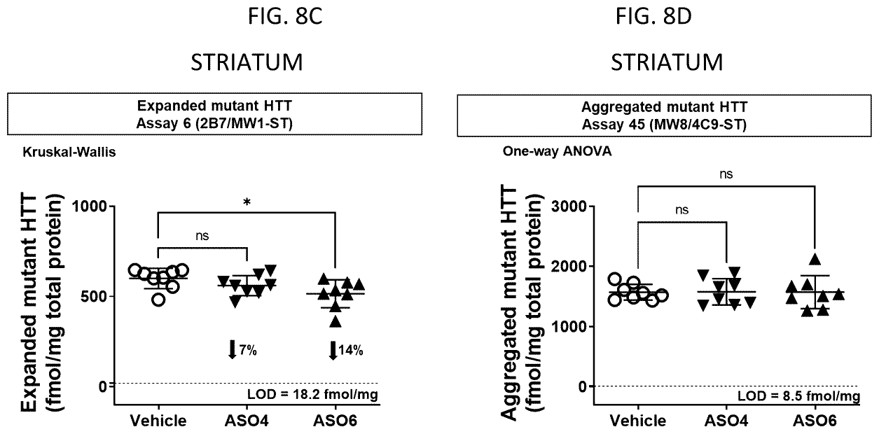

Results: As shown in Tables 9 and 10 above, and in FIGS. 7A and 7B, ASO6 exhibited significant improvements in function in the Total Rearing Frequency and the Total Distance Travelled, and ASO4 exhibited improvement in the Total Rearing Frequency. This improvement in function in the ASO6 treated group correlates well with a reduction of the soluble and aggregated protein in the Cortex (17% and 16% reduction, respectively) and the soluble protein in the striatum of 14% (there was no change in the aggregated protein in the striatum). It is important to note that the ASO6 treated group rearing and total distance traveled is statistically different than the vehicle group, suggesting the ASO6 treatment prevents neurodegeneration that leads to reduced movement behavior. See FIGS. 8A and 8B. The improvement in function for the ASO4 group also correlates well with the aggregated protein in the Cortex. See FIG. 8B.

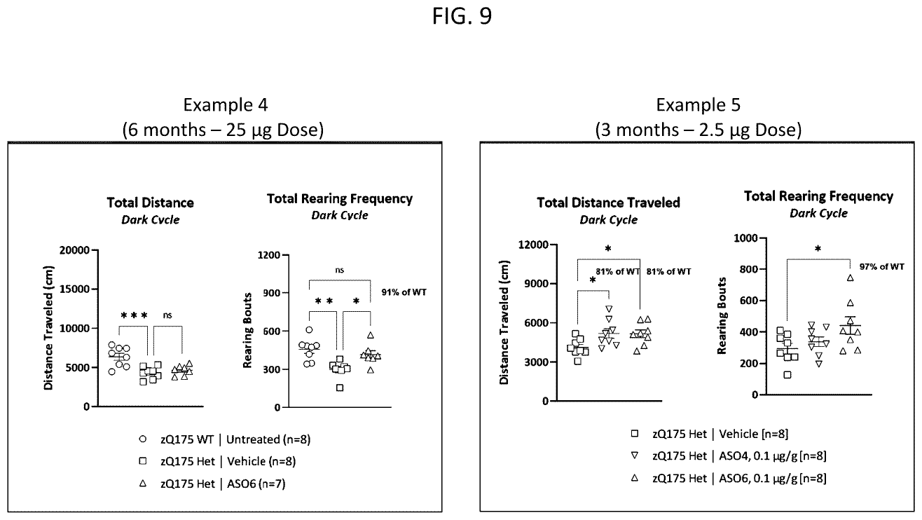

Also, as can be seen from both independent chronic dose response studies, the compositions according to embodiments of the present disclosure exhibit significant improvements in functional measures, demonstrating a reversal of phenotype. For example, as shown in FIG. 9 (which compares results of both studies), in the higher dose (25 μg) chronic dose response study, the ASO6 treated mice showed a Total Rearing Frequency after treatment of 91% of WT and no improvement in Total Distance Traveled. And in the lower dose (2.5 μg) chronic dose response study, the ASO6 treated mice showed a Total Rearing Frequency of 97% of WT in addition to a Total Distance Traveled of 81% of WT.

Additionally, while both dose response studies indicate significant improvements, the lower dose (2.5 μg) surprisingly and unexpectedly improved the function with respect to both Total Distance Travelled and Total Rearing Frequency in the amount of 25% and 50% relative to the vehicle control group, while the high dose (25 μg) only improved the Total Rearing Frequency relative to the vehicle control group in the amount of 39%. Relative to the WT mice, the Total Rearing Frequency was improved by 97% for the lower dose (2.5 μg) and 91% for the higher dose (25 μg)—both demonstrating a reversal of the phenotype of the zQ175 mice back to WT as noted above. But the Total Distance Traveled was surprisingly and unexpectedly improved by 81% relative to the WT group in the lower dose (2.5 μg) study, while no improvement in the Total Distance Travelled relative to WT group (or the vehicle control group) was observed in the high dose (25 μg) study.

Further, while both studies show statistically significant functional improvements and protein knockdown results, a comparison of the two studies surprisingly and unexpectedly shows that the lower dose (2.5 μg) performed better than the higher dose (25 μg). For example, as seen in a comparison of FIGS. 6B and 8B, the lower dose (2.5 μg) exhibited greater reduction of the aggregated protein in the cortex in the ASO6 treated group (statistically significant KD of 16%) compared to no statistically significant KD in the cortex in the high dose (25 μg) ASO6 treated group. Additionally, as seen in FIGS. 6C and 8C, the lower dose (2.5 μg) exhibited greater reduction of the soluble protein in the striatum in the ASO6 treated group (statistically significant KD of 14%) compared to no statistically significant KD in the striatum in the high dose (25 μg) ASO6 treated group. The surprising finding that a dramatically low dose is effective in reducing a target mRNA and protein reduces the risk to patients from general ASO associated toxicity.

As used herein, unless otherwise expressly specified, all numbers such as those expressing values, ranges, amounts or percentages may be read as if prefaced by the word “about,” even if the term does not expressly appear. As used herein, the terms “substantially”, “about”, and similar terms are used as terms of approximation and not as terms of degree, and are intended to account for the inherent deviations in measured or calculated values that would be recognized by those of ordinary skill in the art. Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. As used herein, the terms “combination thereof” and “combinations thereof” may refer to a chemical combination (e.g., an alloy or chemical compound), a mixture, or a solution with multiple solutes.

The terminology used herein is for the purpose of describing particular embodiments only and is not intended to be limiting of the present disclosure. Plural encompasses singular and vice versa. As used herein, the singular forms “a” and “an” are intended to include the plural forms as well, unless the context clearly indicates otherwise. It will be further understood that the terms “comprises”, “comprising”, “includes”, and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any and all combinations of one or more of the associated listed items. Expressions such as “at least one of”, when preceding a list of elements, modify the entire list of elements and do not modify the individual elements of the list.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. A composition for treating Huntington's disease (HD) by reducing endogenous Huntingtin (HTT) protein and/or mRNA levels in a target, the composition comprising:
an anti-sense oligonucleotide (ASO) comprising an ASO sequence selected from SEQ ID NOs:18 to 86 or a variant thereof; and
a carrier peptide comprising a base peptide sequence of SEQ ID NO:1 or a variant thereof.

2. The composition of claim 1, wherein the ASO sequence comprises a common sub-sequence selected from SEQ ID NOs:18 to 28.

3. The composition of claim 1, wherein the ASO sequence is a 20-mer selected from SEQ ID NOs:29 to 86.

4. The composition of claim 1, wherein the ASO sequence is selected from SEQ ID NOs: 87, 97, 99, 100, 125 and 41.

5. The composition of claim 1, wherein the ASO sequence comprises one or more chemical modifications selected from 2′-O-methylation of A, G, C, and/or U, and substitution of phosphorothioate in a phosphodiester backbone unit.

6. The composition of claim 3, wherein the ASO sequence is 2′-O-methylated at positions 1-5 and 16-20, and phosphorothioated between positions 1-3, 6-16, and 18-20.

7. The composition of any one of claims 1-6, wherein the carrier peptide further comprises a linker sequence of SEQ ID NO:14 prepended to the base sequence.

8. The composition of any one of claims 1-6, wherein the carrier peptide is selected from SEQ ID NOs:16 and 17.

9. The composition of any one of claims 1-6, wherein the ASO is conjugated or complexed with the carrier peptide.

10. The composition of any one of claims 1-6, wherein a ratio of the ASO and the carrier peptide is 1:12 to 30:1.

11. A method of treating Huntington's disease (HD) by reducing endogenous Huntingtin (HTT) protein and/or mRNA levels in a target, the method comprising:
administering a composition comprising an ASO comprising an ASO sequence selected from SEQ ID NOs:18 to 86.

12. The method of claim 11, wherein the ASO sequence comprises a common sub-sequence selected from SEQ ID NOs:18 to 28.

13. The method of claim 11, wherein the ASO sequence comprises a 20-mer selected from SEQ ID NOs:29 to 86.

14. The method of claim 11, wherein the ASO sequence is selected from SEQ ID NOs: 87, 97, 99, 100, 125 and 41.

15. The method of any one of claims 11-14, wherein the ASO sequence comprises one or more chemical modifications selected from 2′-O-methylation of A, G, and/or U; and substitution of phosphorothioate in a phosphodiester backbone unit.

16. The method of claim 13, wherein the ASO sequence is 2′-O-methylated at positions 1-5 and 16-20, and phosphorothioated between positions 1-3, 6-16, and 18-20.

17. A method of treating Huntington's disease (HD) by reducing endogenous Huntingtin (HTT) protein and/or HTT mRNA levels in a target, the method comprising:
administering the composition of any one of claims 1-6 to the target.

18. The method of claim 17, wherein the composition is prepared by mixing the ASO or the variant thereof and the carrier peptide or the variant thereof in a ratio of 1:12 to 1:30 in a solution that is acceptable for parenteral administration.

19. The method of claim 18, wherein the solution is a saline solution comprising 0.1 wt % to 1 wt % PEG 8000.

20. The method of claim 18, wherein the solution is a dextrose solution comprising 0.01 wt % to 1.5 wt % Polysorbate 80.

21. The method of claim 17, wherein the administering of the composition occurs in a human subject.

22. The method of claim 17, wherein the target is a cell, and the cell is selected from the group consisting of neurons, neuronal cells, brain cells, glial cells, astrocytes, neuronal supporting cells, and cells of the central nervous system (CNS).

23. The method of claim 17, further comprising conjugating or complexing the ASO to the carrier peptide, prior to administering the composition.

24. The method of claim 23, wherein the carrier peptide further comprises a linker sequence of SEQ ID NO:14 prepended to the base sequence, and
the ASO is conjugated with the carrier peptide via the linker sequence.

25. The method of claim 17, wherein the HTT mRNA levels are decreased by 20% to 90%.

26. A composition for treating Huntington's disease (HD) by reducing endogenous Huntingtin (HTT) protein and/or mRNA levels in a target, the composition comprising:
a 16 to 27-mer anti-sense oligonucleotide (ASO) comprising one of SEQ ID NOs:18 to 28; and
a carrier peptide comprising a base peptide sequence of SEQ ID NO:1 or a variant thereof.

27. The composition of claim 26, wherein the 16 to 27-mer ASO is complementary to a HTT mRNA sequence.

28. The composition of claim 26, wherein the ASO is an 18 to 24-mer.

29. The composition of claim 26, wherein the ASO is a 20-mer.

30. The composition of any one of claims 26 to 29, wherein the ASO sequence comprises one or more chemical modifications selected from 2′-O-methylation of A, G, C, and/or U, and substitution of phosphorothioate in a phosphodiester backbone unit.

31. The composition of any one of claims 26 to 29, wherein the carrier peptide further comprises a linker sequence of SEQ ID NO:14, prepended to the base sequence.

32. The composition of any one of claims 26 to 29, wherein the carrier peptide is selected from SEQ ID NOs:16 and 17.

33. The composition of claim 31, wherein the ASO is conjugated with the carrier peptide via the linker sequence.

34. The method of any of claims 11-25, wherein the administration comprises administering the composition in a dose sufficient to deliver an amount of the ASO of lower than 100 μg per kg, or lower than 4,000 μg per kg, or lower than 1,000 μg per kg.

35. The method of any of claims 11-25 and 34, wherein the administration comprises administering the composition in a dose sufficient to deliver an amount of the ASO of 1 μg per kg to 4,000 μg per kg.

36. The method of any of claims 11-25, 34 and 35, wherein the administration comprises administering the composition in a dose sufficient to deliver an amount of the ASO of 1 μg per kg to 4,000 μg per kg, 1 μg per kg to 3,000 μg per kg, 1 μg per kg to 2,000 μg per kg, 1 μg per kg to 1,000 μg per kg, 1 μg per kg to 600 μg per kg, 2 μg per kg to 4,000 μg per kg, 2 μg per kg to 3,000 μg per kg, 2 μg per kg to 2,000 μg per kg, 2 μg per kg to 1,000 μg per kg, 2 μg per kg to 600 μg per kg, 10 μg per kg to 4,000 μg per kg, 10 μg per kg to 3,000 μg per kg, 10 μg per kg to 2,000 μg per kg, 10 μg per kg to 1,000 μg per kg, 10 μg per kg to 600 μg per kg, 20 μg per kg to 4,000 μg per kg, 20 μg per kg to 3,000 μg per kg, 20 μg per kg to 2,000 μg per kg, 20 μg per kg to 1,000 μg per kg, 20 μg per kg to 600 μg per kg, 40 μg per kg to 4,000 μg per kg, 40 μg per kg to 3,000 μg per kg, 40 μg per kg to 2,000 μg per kg, 40 μg per kg to 1,000 μg per kg, 40 μg per kg to 600 μg per kg, 100 μg per kg to 4,000 μg per kg, 100 μg per kg to 3,000 μg per kg, 100 μg per kg to 2,000 μg per kg, 100 μg per kg to 1,000 μg per kg, 100 μg per kg to 600 μg per kg, 200 μg per kg to 4,000 μg per kg, 200 μg per kg to 3,000 μg per kg, 200 μg per kg to 2,000 μg per kg, 200 μg per kg to 1,000 μg per kg, 200 μg per kg to 600 μg per kg, 350 μg per kg to 4,000 μg per kg, 350 μg per kg to 3,000 μg per kg, 350 μg per kg to 2,000 μg per kg, 350 μg per kg to 1,000 μg per kg, 350 μg per kg to 600 μg per kg, 1 μg per kg to 350 μg per kg, 2 μg per kg to 350 μg per kg, 10 μg per kg to 350 μg per kg, 20 μg per kg to 350 μg per kg, 40 μg per kg to 350 μg per kg, 100 μg per kg to 350 μg per kg, 200 μg per kg to 350 μg per kg, 1 μg per kg to 200 μg per kg, 2 μg per kg to 200 μg per kg, 10 μg per kg to 200 μg per kg, 20 μg per kg to 200 μg per kg, 40 μg per kg to 200 μg per kg, 1 μg per kg to 100 μg per kg, 2 μg per kg to 100 μg per kg, 10 μg per kg to 100 μg per kg, 20 μg per kg to 100 μg per kg, 40 μg per kg to 100 μg per kg, 1 μg per kg to 50 μg per kg, 2 μg per kg to 50 μg per kg, 10 μg per kg to 50 μg per kg, 20 μg per kg to 50 μg per kg, 40 μg per kg to 50 μg per kg, 1 μg per kg to 40 μg per kg, 2 μg per kg to 40 μg per kg, 10 μg per kg to 40 μg per kg, 20 μg per kg to 40 μg per kg, 1 μg per kg to 20 μg per kg, 2 μg per kg to 20 μg per kg, 10 μg per kg to 20 μg per kg, 1 μg per kg to 10 μg per kg, 2 μg per kg to 10 μg per kg.

37. The method of any of claims 11-25 and 34-36, wherein the administration of the composition comprises administering the composition in a dose sufficient to deliver an amount of the ASO of 1 μg per kg, 2 μg per kg, 10 μg per kg, 20 μg per kg, 40 μg per kg, 100 μg per kg, 200 μg per kg, 350 μg per kg, 600 μg per kg, 1,000 μg per kg, or 2000 μg per kg.

38. The method of any of claims 11-25, wherein the administration of the composition comprises administering the composition in a dose sufficient to deliver an amount of the ASO of 1.7 μg per kg to 6,800 μg per kg.

39. The method of any of claims 11-25 and 38, wherein the administration of the composition comprises administering the composition in a dose sufficient to deliver an amount of the ASO of 1.7 μg per kg to 6,800 μg per kg, 1.7 μg per kg to 5,100 μg per kg, 1.7 μg per kg to 3,400 μg per kg, 1.7 μg per kg to 1,700 μg per kg, 1.7 μg per kg to 1,000 μg per kg, 3.4 μg per kg to 6,800 μg per kg, 3.4 μg per kg to 5,100 μg per kg, 3.4 μg per kg to 3,400 μg per kg, 3.4 μg per kg to 1,700 μg per kg, 3.4 μg per kg to 1,000 μg per kg, 17 μg per kg to 6,800 μg per kg, 17 μg per kg to 5,100 μg per kg, 17 μg per kg to 3,400 μg per kg, 17 μg per kg to 1,700 μg per kg, 17 μg per kg to 1,000 μg per kg, 34 μg per kg to 6,800 μg per kg, 34 μg per kg to 5,100 μg per kg, 34 μg per kg to 3,400 μg per kg, 34 μg per kg to 1,700 μg per kg, 34 μg per kg to 1,000 μg per kg, 68 μg per kg to 6,800 μg per kg, 68 μg per kg to 5,100 μg per kg, 68 μg per kg to 3,400 μg per kg, 68 μg per kg to 1,700 μg per kg, 68 μg per kg to 1,000 μg per kg, 170 μg per kg to 6,800 μg per kg, 170 μg per kg to 6,800 μg per kg, 170 μg per kg to 3,400 μg per kg, 170 μg per kg to 1,700 μg per kg, 170 μg per kg to 1,000 μg per kg, 340 μg per kg to 6,800 μg per kg, 340 μg per kg to 5,100 μg per kg, 340 μg per kg to 3,400 μg per kg, 340 μg per kg to 1,700 μg per kg, 340 μg per kg to 1,000 μg per kg, 595 μg per kg to 6,800 μg per kg, 595 μg per kg to 6,800 μg per kg, 595 μg per kg to 6,800 μg per kg, 595 μg per kg to 1,700 μg per kg, 595 μg per kg to 1,000 μg per kg, 1.7 μg per kg to 595 μg per kg, 3.4 μg per kg to 595 μg per kg, 17 μg per kg to 595 μg per kg, 34 μg per kg to 595 μg per kg, 68 μg per kg to 595 μg per kg, 170 μg per kg to 595 μg per kg, 340 μg per kg to 595 μg per kg, 1.7 μg per kg to 340 μg per kg, 3.4 μg per kg to 340 μg per kg, 17 μg per kg to 340 μg per kg, 34 μg per kg to 340 μg per kg, 68 μg per kg to 340 μg per kg, 1.7 μg per kg to 85 μg per kg, 3.4 μg per kg to 85 μg per kg, 3.4 μg per kg to 85 μg per kg, 34 μg per kg to 85 μg per kg, 68 μg per kg to 85 μg per kg, 1.7 μg per kg to 68 μg per kg, 3.4 μg per kg to 68 μg per kg, 17 μg per kg to 68 μg per kg, 34 μg per kg to 68 μg per kg, 1.7 μg per kg to 34 μg per kg, 3.4 μg per kg to 34 μg per kg, 17 μg per kg to 34 μg per kg, 1.7 μg per kg to 17 μg per kg, 3.4 μg per kg to 17 μg per kg.

40. The method of any of claims 11-25, 38 and 39, wherein the administration of the composition comprises administering the composition in a dose sufficient to deliver an amount of the ASO of 1.7 μg per kg, 3.4 μg per kg, 17 μg per kg, 34 μg per kg, 68 μg per kg, 85 μg per kg, 170 μg per kg, 340 μg per kg, 595 μg per kg, 1,700 μg per kg, 1,000 μg per kg, or 3,400 μg per kg.

41. The method of any of claims 11-40, wherein the administering the composition comprises administering the composition to a human or animal subject diagnosed with, or suffering one or more symptoms of Huntington's disease.


SEQUENCE LISTING (153 sequences)

SEQ ID NO: 1 - skipped

SEQ ID NO: 2 - protein, 16 aa, synthetic construct
MTTRTYFTPYRMKVRK

SEQ ID NO: 3 - protein, 16 aa, synthetic construct
MTTRTYYTPTRMKVSK

SEQ ID NO: 4 - protein, 17 aa, synthetic construct
MTWCDYFTPSRGKVRKS

SEQ ID NO: 5 - protein, 16 aa, synthetic construct
MTTRTYFTPYRGKVRK

SEQ ID NO: 6 - protein, 17 aa, synthetic construct
MTTRTYATPYRMKVRKS

SEQ ID NO: 7 - protein, 17 aa, synthetic construct
MTTRTYFTPYAMADRKS

SEQ ID NO: 8 - protein, 10 aa, synthetic construct
MPENPRPGTP

SEQ ID NO: 9 - protein, 26 aa, synthetic construct
  VARIANT 1: note = M or T
  VARIANT 2: note = T or I
  VARIANT 3: note = T or W
  VARIANT 14: note = R or C
  VARIANT 15: note = T or D
  VARIANT 16: note = Y, I, or G
  VARIANT 17: note = F or Y
  VARIANT 18: note = T or C
  VARIANT 19: note = P, N, or S
  VARIANT 20: note = Y, T, or S
  VARIANT 22: note = M or G
  VARIANT 24: note = V or R
  VARIANT 25: note = R, S, A, or I
  VARIANT 26: note = K, S, or D
XXXMPENPRPGTPXXXXXXXRXKXXX

SEQ ID NO: 10 - protein, 27 aa, synthetic construct
MTTMPENPRPGTPRTYFTPYRMKVRKS

SEQ ID NO: 11-13 - skipped (3 entries)

SEQ ID NO: 14 - protein, 16 aa, synthetic construct
  VARIANT 2: note = Gly or absent
  VARIANT 3: note = Gly or absent
  VARIANT 4: note = Gly or absent
  VARIANT 5..16: note = Any standard amino acid or absent, provided that at least three residues are presnet and are selected from Arg and Lys
GGGGXXXXXXXXXXXX

SEQ ID NO: 15 - protein, 13 aa, synthetic construct
  VARIANT 5: note = Arg or Lys
  VARIANT 6: note = Arg or Lys
  VARIANT 7: note = Arg or Lys
  VARIANT 8: note = Arg or Lys
  VARIANT 9: note = Arg or Lys
  VARIANT 10: note = Arg or Lys
  VARIANT 11: note = Arg or Lys
  VARIANT 12: note = Arg or Lys
  VARIANT 13: note = Arg or Lys
  VARIANT 5..13: note = This region may encompass 7-9 residues
GGGGXXXXXXXXX

SEQ ID NO: 16 - protein, 30 aa, synthetic construct
MTWCDYFTPSRGKVRKSGGGGRRRRRRRRR

SEQ ID NO: 17 - protein, 28 aa, synthetic construct
MTWCDYFTPSRGKVRKSGGGGRRRRRRR

SEQ ID NO: 18 - DNA, 11 bp, synthetic construct
  source 1..11: mol_type = other DNA
ttcacacggtc

SEQ ID NO: 19 - DNA, 17 bp, synthetic construct
  source 1..17: mol_type = other DNA
agttcataaacctggac

SEQ ID NO: 20 - DNA, 17 bp, synthetic construct
  source 1..17: mol_type = other DNA
aacacaatttcagaact

SEQ ID NO: 21 - DNA, 14 bp, synthetic construct
  source 1..14: mol_type = other DNA
tcgatgtagttcaa

SEQ ID NO: 22 - skipped

SEQ ID NO: 23 - DNA, 17 bp, synthetic construct
  source 1..17: mol_type = other DNA
tcattggttctcgacta

SEQ ID NO: 24 - DNA, 17 bp, synthetic construct
  source 1..17: mol_type = other DNA
ctgatggtactggatga

SEQ ID NO: 25 - DNA, 12 bp, synthetic construct
  source 1..12: mol_type = other DNA
acataatcacag

SEQ ID NO: 26 - DNA, 16 bp, synthetic construct
  source 1..16: mol_type = other DNA
aatgatggagggggtg

SEQ ID NO: 27 - DNA, 13 bp, synthetic construct
  source 1..13: mol_type = other DNA
ctggtgtctgtag

SEQ ID NO: 28 - DNA, 13 bp, synthetic construct
  source 1..13: mol_type = other DNA
ctctttactgata

SEQ ID NO: 29 - DNA, 20 bp, synthetic construct
  source 1..20: mol_type = other DNA
ttcacacggtctttcttggt

SEQ ID NO: 30 - DNA, 20 bp, synthetic construct
  source 1..20: mol_type = other DNA
attcacacggtctttcttgg

SEQ ID NO: 31 - DNA, 20 bp, synthetic construct
  source 1..20: mol_type = other DNA
gattcacacggtctttcttg

SEQ ID NO: 32 - DNA, 20 bp, synthetic construct
  source 1..20: mol_type = other DNA
tgattcacacggtctttctt

SEQ ID NO: 33 - DNA, 20 bp, synthetic construct
  source 1..20: mol_type = other DNA
atgattcacacggtctttct

SEQ ID NO: 34 - DNA, 20 bp, synthetic construct
  source 1..20: mol_type = other DNA
aatgattcacacggtctttc

SEQ ID NO: 35 - DNA, 20 bp, synthetic construct
  source 1..20: mol_type = other DNA
caatgattcacacggtcttt

SEQ ID NO: 36 - DNA, 20 bp, synthetic construct
  source 1..20: mol_type = other DNA
acaatgattcacacggtctt

SEQ ID NO: 37 - DNA, 20 bp, synthetic construct
  source 1..20: mol_type = other DNA
gacaatgattcacacggtct

SEQ ID NO: 38 - DNA, 20 bp, synthetic construct
  source 1..20: mol_type = other DNA
agacaatgattcacacggtc

SEQ ID NO: 39 - DNA, 20 bp, synthetic construct
  source 1..20: mol_type = other DNA
agttcataaacctggacaag

SEQ ID NO: 40 - DNA, 20 bp, synthetic construct
  source 1..20: mol_type = other DNA
cagttcataaacctggacaa

SEQ ID NO: 41 - DNA, 20 bp, synthetic construct
  source 1..20: mol_type = other DNA
gtcagttcataaacctggac

SEQ ID NO: 42 - DNA, 20 bp, synthetic construct
  source 1..20: mol_type = other DNA
aacacaatttcagaactgtc

SEQ ID NO: 43 - DNA, 20 bp, synthetic construct
  source 1..20: mol_type = other DNA
ctaacacaatttcagaactg

SEQ ID NO: 44 - DNA, 20 bp, synthetic construct
  source 1..20: mol_type = other DNA
tctaacacaatttcagaact

SEQ ID NO: 45 - DNA, 20 bp, synthetic construct
  source 1..20: mol_type = other DNA
tcgatgtagttcaagatgtc

SEQ ID NO: 46 - DNA, 20 bp, synthetic construct
  source 1..20: mol_type = other DNA
atcgatgtagttcaagatgt

SEQ ID NO: 47 - DNA, 20 bp, synthetic construct
  source 1..20: mol_type = other DNA
gatcgatgtagttcaagatg

SEQ ID NO: 48 - DNA, 20 bp, synthetic construct
  source 1..20: mol_type = other DNA
tgatcgatgtagttcaagat

SEQ ID NO: 49 - DNA, 20 bp, synthetic construct
  source 1..20: mol_type = other DNA
atgatcgatgtagttcaaga

SEQ ID NO: 50 - DNA, 20 bp, synthetic construct
  source 1..20: mol_type = other DNA
catgatcgatgtagttcaag

SEQ ID NO: 51 - DNA, 20 bp, synthetic construct
  source 1..20: mol_type = other DNA
ccatgatcgatgtagttcaa

SEQ ID NO: 52 - DNA, 20 bp, synthetic construct
  source 1..20: mol_type = other DNA
ggggggtttgttagagaagg

SEQ ID NO: 53 - DNA, 20 bp, synthetic construct
  source 1..20: mol_type = other DNA
aggggggtttgttagagaag

SEQ ID NO: 54 - DNA, 20 bp, synthetic construct
  source 1..20: mol_type = other DNA
aaggggggtttgttagagaa

SEQ ID NO: 55 - DNA, 20 bp, synthetic construct
  source 1..20: mol_type = other DNA
gaaggggggtttgttagaga

SEQ ID NO: 56 - DNA, 20 bp, synthetic construct
  source 1..20: mol_type = other DNA
agaaggggggtttgttagag

SEQ ID NO: 57 - DNA, 20 bp, synthetic construct
  source 1..20: mol_type = other DNA
agagaaggggggtttgttag

SEQ ID NO: 58 - DNA, 20 bp, synthetic construct
  source 1..20: mol_type = other DNA
tagagaaggggggtttgtta

SEQ ID NO: 59 - DNA, 20 bp, synthetic construct
  source 1..20: mol_type = other DNA
ttagagaaggggggtttgtt

SEQ ID NO: 60 - DNA, 20 bp, synthetic construct
  source 1..20: mol_type = other DNA
cttagagaaggggggtttgt

SEQ ID NO: 61 - DNA, 20 bp, synthetic construct
  source 1..20: mol_type = other DNA
acttagagaaggggggtttg

SEQ ID NO: 62 - DNA, 20 bp, synthetic construct
  source 1..20: mol_type = other DNA
gacttagagaaggggggttt

SEQ ID NO: 63 - DNA, 20 bp, synthetic construct
  source 1..20: mol_type = other DNA
ggacttagagaaggggggtt

SEQ ID NO: 64 - DNA, 20 bp, synthetic construct
  source 1..20: mol_type = other DNA
tcattggttctcgactaaag

SEQ ID NO: 65 - DNA, 20 bp, synthetic construct
  source 1..20: mol_type = other DNA
atcattggttctcgactaaa

SEQ ID NO: 66 - DNA, 20 bp, synthetic construct
  source 1..20: mol_type = other DNA
catcattggttctcgactaa

SEQ ID NO: 67 - DNA, 20 bp, synthetic construct
  source 1..20: mol_type = other DNA
ccatcattggttctcgacta

SEQ ID NO: 68 - DNA, 20 bp, synthetic construct
  source 1..20: mol_type = other DNA
ctgatggtactggatgagtc

SEQ ID NO: 69 - DNA, 20 bp, synthetic construct
  source 1..20: mol_type = other DNA
cacctgatggtactggatga

SEQ ID NO: 70 - DNA, 20 bp, synthetic construct
  source 1..20: mol_type = other DNA
acataatcacagaagagaat

SEQ ID NO: 71 - DNA, 20 bp, synthetic construct
  source 1..20: mol_type = other DNA
gacagacataatcacagaag

SEQ ID NO: 72 - DNA, 20 bp, synthetic construct
  source 1..20: mol_type = other DNA
ctgacagacataatcacaga

SEQ ID NO: 73 - DNA, 20 bp, synthetic construct
  source 1..20: mol_type = other DNA
tctgacagacataatcacag

SEQ ID NO: 74 - DNA, 20 bp, synthetic construct
  source 1..20: mol_type = other DNA
gtcaccaggacaccaaggag

SEQ ID NO: 75 - DNA, 20 bp, synthetic construct
  source 1..20: mol_type = other DNA
aatgatggagggggtggact

SEQ ID NO: 76 - DNA, 20 bp, synthetic construct
  source 1..20: mol_type = other DNA
gtaaatgatggagggggtgg

SEQ ID NO: 77 - DNA, 20 bp, synthetic construct
  source 1..20: mol_type = other DNA
ggtaaatgatggagggggtg

SEQ ID NO: 78 - DNA, 20 bp, synthetic construct
  source 1..20: mol_type = other DNA
ctggtgtctgtagaagtctg

SEQ ID NO: 79 - DNA, 20 bp, synthetic construct
  source 1..20: mol_type = other DNA
tatctggtgtctgtagaagt

SEQ ID NO: 80 - DNA, 20 bp, synthetic construct
  source 1..20: mol_type = other DNA
ctctatctggtgtctgtaga

SEQ ID NO: 81 - DNA, 20 bp, synthetic construct
  source 1..20: mol_type = other DNA
cctctatctggtgtctgtag

SEQ ID NO: 82 - DNA, 20 bp, synthetic construct
  source 1..20: mol_type = other DNA
ctctttactgatataattaa

SEQ ID NO: 83 - DNA, 20 bp, synthetic construct
  source 1..20: mol_type = other DNA
tctctttactgatataatta

SEQ ID NO: 84 - DNA, 20 bp, synthetic construct
  source 1..20: mol_type = other DNA
aatctctttactgatataat

SEQ ID NO: 85 - DNA, 20 bp, synthetic construct
  source 1..20: mol_type = other DNA
taatctctttactgatataa

SEQ ID NO: 86 - DNA, 20 bp, synthetic construct
  source 1..20: mol_type = other DNA
aattaatctctttactgata

SEQ ID NO: 87 - DNA, 20 bp, synthetic construct
  source 1..20: mol_type = other DNA
  misc_feature 1..5: note = RNA
  misc_feature 6..15: note = DNA
  misc_feature 16..20: note = RNA
ttcacacggtctttcttggt

SEQ ID NO: 88 - DNA, 20 bp, synthetic construct
  source 1..20: mol_type = other DNA
  misc_feature 1..5: note = RNA
  misc_feature 6..15: note = DNA
  misc_feature 16..20: note = RNA
attcacacggtctttcttgg

SEQ ID NO: 89 - DNA, 20 bp, synthetic construct
  source 1..20: mol_type = other DNA
  misc_feature 1..5: note = RNA
  misc_feature 6..15: note = DNA
  misc_feature 16..20: note = RNA
gattcacacggtctttcttg

SEQ ID NO: 90 - DNA, 20 bp, synthetic construct
  source 1..20: mol_type = other DNA
  misc_feature 1..5: note = RNA
  misc_feature 6..15: note = DNA
  misc_feature 16..20: note = RNA
tgattcacacggtctttctt

SEQ ID NO: 91 - DNA, 20 bp, synthetic construct
  source 1..20: mol_type = other DNA
  misc_feature 1..5: note = RNA
  misc_feature 6..15: note = DNA
  misc_feature 16..20: note = RNA
atgattcacacggtctttct

SEQ ID NO: 92 - DNA, 20 bp, synthetic construct
  source 1..20: mol_type = other DNA
  misc_feature 1..5: note = RNA
  misc_feature 6..15: note = DNA
  misc_feature 16..20: note = RNA
aatgattcacacggtctttc

SEQ ID NO: 93 - DNA, 20 bp, synthetic construct
  source 1..20: mol_type = other DNA
  misc_feature 1..5: note = RNA
  misc_feature 6..15: note = DNA
  misc_feature 16..20: note = RNA
caatgattcacacggtcttt

SEQ ID NO: 94 - DNA, 20 bp, synthetic construct
  source 1..20: mol_type = other DNA
  misc_feature 1..5: note = RNA
  misc_feature 6..15: note = DNA
  misc_feature 16..20: note = RNA
acaatgattcacacggtctt

SEQ ID NO: 95 - DNA, 20 bp, synthetic construct
  source 1..20: mol_type = other DNA
  misc_feature 1..5: note = RNA
  misc_feature 6..15: note = DNA
  misc_feature 16..20: note = RNA
gacaatgattcacacggtct

SEQ ID NO: 96 - DNA, 20 bp, synthetic construct
  source 1..20: mol_type = other DNA
  misc_feature 1..5: note = RNA
  misc_feature 6..15: note = DNA
  misc_feature 16..20: note = RNA
agacaatgattcacacggtc

SEQ ID NO: 97 - DNA, 20 bp, synthetic construct
  source 1..20: mol_type = other DNA
  misc_feature 1..5: note = RNA
  misc_feature 6..15: note = DNA
  misc_feature 16..20: note = RNA
agttcataaacctggacaag

SEQ ID NO: 98 - DNA, 20 bp, synthetic construct
  source 1..20: mol_type = other DNA
  misc_feature 1..5: note = RNA
  misc_feature 6..15: note = DNA
  misc_feature 16..20: note = RNA
cagttcataaacctggacaa

SEQ ID NO: 99 - DNA, 20 bp, synthetic construct
  source 1..20: mol_type = other DNA
  misc_feature 1..5: note = RNA
  misc_feature 6..15: note = DNA
  misc_feature 16..20: note = RNA
gtcagttcataaacctggac

SEQ ID NO: 100 - DNA, 20 bp, synthetic construct
  source 1..20: mol_type = other DNA
  misc_feature 1..5: note = RNA
  misc_feature 6..15: note = DNA
  misc_feature 16..20: note = RNA
aacacaatttcagaactgtc

SEQ ID NO: 101 - DNA, 20 bp, synthetic construct
  source 1..20: mol_type = other DNA
  misc_feature 1..5: note = RNA
  misc_feature 6..15: note = DNA
  misc_feature 16..20: note = RNA
ctaacacaatttcagaactg

SEQ ID NO: 102 - DNA, 20 bp, synthetic construct
  source 1..20: mol_type = other DNA
  misc_feature 1..5: note = RNA
  misc_feature 6..15: note = DNA
  misc_feature 16..20: note = RNA
tctaacacaatttcagaact

SEQ ID NO: 103 - DNA, 20 bp, synthetic construct
  source 1..20: mol_type = other DNA
  misc_feature 1..5: note = RNA
  misc_feature 6..15: note = DNA
  misc_feature 16..20: note = RNA
tcgatgtagttcaagatgtc

SEQ ID NO: 104 - DNA, 20 bp, synthetic construct
  source 1..20: mol_type = other DNA
  misc_feature 1..5: note = RNA
  misc_feature 6..15: note = DNA
  misc_feature 16..20: note = RNA
atcgatgtagttcaagatgt

SEQ ID NO: 105 - DNA, 20 bp, synthetic construct
  source 1..20: mol_type = other DNA
  misc_feature 1..5: note = RNA
  misc_feature 6..15: note = DNA
  misc_feature 16..20: note = RNA
gatcgatgtagttcaagatg

SEQ ID NO: 106 - DNA, 20 bp, synthetic construct
  source 1..20: mol_type = other DNA
  misc_feature 1..5: note = RNA
  misc_feature 6..15: note = DNA
  misc_feature 16..20: note = RNA
tgatcgatgtagttcaagat

SEQ ID NO: 107 - DNA, 20 bp, synthetic construct
  source 1..20: mol_type = other DNA
  misc_feature 1..5: note = RNA
  misc_feature 6..15: note = DNA
  misc_feature 16..20: note = RNA
atgatcgatgtagttcaaga

SEQ ID NO: 108 - DNA, 20 bp, synthetic construct
  source 1..20: mol_type = other DNA
  misc_feature 1..5: note = RNA
  misc_feature 6..15: note = DNA
  misc_feature 16..20: note = RNA
catgatcgatgtagttcaag

SEQ ID NO: 109 - DNA, 20 bp, synthetic construct
  source 1..20: mol_type = other DNA
  misc_feature 1..5: note = RNA
  misc_feature 6..15: note = DNA
  misc_feature 16..20: note = RNA
ccatgatcgatgtagttcaa

SEQ ID NO: 110 - DNA, 20 bp, synthetic construct
  source 1..20: mol_type = other DNA
  misc_feature 1..5: note = RNA
  misc_feature 6..15: note = DNA
  misc_feature 16..20: note = RNA
ggggggtttgttagagaagg

SEQ ID NO: 111 - DNA, 20 bp, synthetic construct
  source 1..20: mol_type = other DNA
  misc_feature 1..5: note = RNA
  misc_feature 6..15: note = DNA
  misc_feature 16..20: note = RNA
aggggggtttgttagagaag

SEQ ID NO: 112 - DNA, 20 bp, synthetic construct
  source 1..20: mol_type = other DNA
  misc_feature 1..5: note = RNA
  misc_feature 6..15: note = DNA
  misc_feature 16..20: note = RNA
aaggggggtttgttagagaa

SEQ ID NO: 113 - DNA, 20 bp, synthetic construct
  source 1..20: mol_type = other DNA
  misc_feature 1..5: note = RNA
  misc_feature 6..15: note = DNA
  misc_feature 16..20: note = RNA
gaaggggggtttgttagaga

SEQ ID NO: 114 - DNA, 20 bp, synthetic construct
  source 1..20: mol_type = other DNA
  misc_feature 1..5: note = RNA
  misc_feature 6..15: note = DNA
  misc_feature 16..20: note = RNA
agaaggggggtttgttagag

SEQ ID NO: 115 - DNA, 20 bp, synthetic construct
  source 1..20: mol_type = other DNA
  misc_feature 1..5: note = RNA
  misc_feature 6..15: note = DNA
  misc_feature 16..20: note = RNA
agagaaggggggtttgttag

SEQ ID NO: 116 - DNA, 20 bp, synthetic construct
  source 1..20: mol_type = other DNA
  misc_feature 1..5: note = RNA
  misc_feature 6..15: note = DNA
  misc_feature 16..20: note = RNA
tagagaaggggggtttgtta

SEQ ID NO: 117 - DNA, 20 bp, synthetic construct
  source 1..20: mol_type = other DNA
  misc_feature 1..5: note = RNA
  misc_feature 6..15: note = DNA
  misc_feature 16..20: note = RNA
ttagagaaggggggtttgtt

SEQ ID NO: 118 - DNA, 20 bp, synthetic construct
  source 1..20: mol_type = other DNA
  misc_feature 1..5: note = RNA
  misc_feature 6..15: note = DNA
  misc_feature 16..20: note = RNA
cttagagaaggggggtttgt

SEQ ID NO: 119 - DNA, 20 bp, synthetic construct
  source 1..20: mol_type = other DNA
  misc_feature 1..5: note = RNA
  misc_feature 6..15: note = DNA
  misc_feature 16..20: note = RNA
acttagagaaggggggtttg

SEQ ID NO: 120 - DNA, 20 bp, synthetic construct
  source 1..20: mol_type = other DNA
  misc_feature 1..5: note = RNA
  misc_feature 6..15: note = DNA
  misc_feature 16..20: note = RNA
gacttagagaaggggggttt

SEQ ID NO: 121 - DNA, 20 bp, synthetic construct
  source 1..20: mol_type = other DNA
  misc_feature 1..5: note = RNA
  misc_feature 6..15: note = DNA
  misc_feature 16..20: note = RNA
ggacttagagaaggggggtt

SEQ ID NO: 122 - DNA, 20 bp, synthetic construct
  source 1..20: mol_type = other DNA
  misc_feature 1..5: note = RNA
  misc_feature 6..15: note = DNA
  misc_feature 16..20: note = RNA
tcattggttctcgactaaag

SEQ ID NO: 123 - DNA, 20 bp, synthetic construct
  source 1..20: mol_type = other DNA
  misc_feature 1..5: note = RNA
  misc_feature 6..15: note = DNA
  misc_feature 16..20: note = RNA
atcattggttctcgactaaa

SEQ ID NO: 124 - DNA, 20 bp, synthetic construct
  source 1..20: mol_type = other DNA
  misc_feature 1..5: note = RNA
  misc_feature 6..15: note = DNA
  misc_feature 16..20: note = RNA
catcattggttctcgactaa

SEQ ID NO: 125 - DNA, 20 bp, synthetic construct
  source 1..20: mol_type = other DNA
  misc_feature 1..5: note = RNA
  misc_feature 6..15: note = DNA
  misc_feature 16..20: note = RNA
ccatcattggttctcgacta

SEQ ID NO: 126 - DNA, 20 bp, synthetic construct
  source 1..20: mol_type = other DNA
  misc_feature 1..5: note = RNA
  misc_feature 6..15: note = DNA
  misc_feature 16..20: note = RNA
ctgatggtactggatgagtc

SEQ ID NO: 127 - DNA, 20 bp, synthetic construct
  source 1..20: mol_type = other DNA
  misc_feature 1..5: note = RNA
  misc_feature 6..15: note = DNA
  misc_feature 16..20: note = RNA
cacctgatggtactggatga

SEQ ID NO: 128 - DNA, 20 bp, synthetic construct
  source 1..20: mol_type = other DNA
  misc_feature 1..5: note = RNA
  misc_feature 6..15: note = DNA
  misc_feature 16..20: note = RNA
acataatcacagaagagaat

SEQ ID NO: 129 - DNA, 20 bp, synthetic construct
  source 1..20: mol_type = other DNA
  misc_feature 1..5: note = RNA
  misc_feature 6..15: note = DNA
  misc_feature 16..20: note = RNA
gacagacataatcacagaag

SEQ ID NO: 130 - DNA, 20 bp, synthetic construct
  source 1..20: mol_type = other DNA
  misc_feature 1..5: note = RNA
  misc_feature 6..15: note = DNA
  misc_feature 16..20: note = RNA
ctgacagacataatcacaga

SEQ ID NO: 131 - DNA, 20 bp, synthetic construct
  source 1..20: mol_type = other DNA
  misc_feature 1..5: note = RNA
  misc_feature 6..15: note = DNA
  misc_feature 16..20: note = RNA
tctgacagacataatcacag

SEQ ID NO: 132 - DNA, 20 bp, synthetic construct
  source 1..20: mol_type = other DNA
  misc_feature 1..5: note = RNA
  misc_feature 6..15: note = DNA
  misc_feature 16..20: note = RNA
gtcaccaggacaccaaggag

SEQ ID NO: 133 - DNA, 20 bp, synthetic construct
  source 1..20: mol_type = other DNA
  misc_feature 1..5: note = RNA
  misc_feature 6..15: note = DNA
  misc_feature 16..20: note = RNA
aatgatggagggggtggact

SEQ ID NO: 134 - DNA, 20 bp, synthetic construct
  source 1..20: mol_type = other DNA
  misc_feature 1..5: note = RNA
  misc_feature 6..15: note = DNA
  misc_feature 16..20: note = RNA
gtaaatgatggagggggtgg

SEQ ID NO: 135 - DNA, 20 bp, synthetic construct
  source 1..20: mol_type = other DNA
  misc_feature 1..5: note = RNA
  misc_feature 6..15: note = DNA
  misc_feature 16..20: note = RNA
ggtaaatgatggagggggtg

SEQ ID NO: 136 - DNA, 20 bp, synthetic construct
  source 1..20: mol_type = other DNA
  misc_feature 1..5: note = RNA
  misc_feature 6..15: note = DNA
  misc_feature 16..20: note = RNA
ctggtgtctgtagaagtctg

SEQ ID NO: 137 - DNA, 20 bp, synthetic construct
  source 1..20: mol_type = other DNA
  misc_feature 1..5: note = RNA
  misc_feature 6..15: note = DNA
  misc_feature 16..20: note = RNA
tatctggtgtctgtagaagt

SEQ ID NO: 138 - DNA, 20 bp, synthetic construct
  source 1..20: mol_type = other DNA
  misc_feature 1..5: note = RNA
  misc_feature 6..15: note = DNA
  misc_feature 16..20: note = RNA
ctctatctggtgtctgtaga

SEQ ID NO: 139 - DNA, 20 bp, synthetic construct
  source 1..20: mol_type = other DNA
  misc_feature 1..5: note = RNA
  misc_feature 6..15: note = DNA
  misc_feature 16..20: note = RNA
cctctatctggtgtctgtag

SEQ ID NO: 140 - DNA, 20 bp, synthetic construct
  source 1..20: mol_type = other DNA
  misc_feature 1..5: note = RNA
  misc_feature 6..15: note = DNA
  misc_feature 16..20: note = RNA
ctctttactgatataattaa

SEQ ID NO: 141 - DNA, 20 bp, synthetic construct
  source 1..20: mol_type = other DNA
  misc_feature 1..5: note = RNA
  misc_feature 6..15: note = DNA
  misc_feature 16..20: note = RNA
tctctttactgatataatta

SEQ ID NO: 142 - DNA, 20 bp, synthetic construct
  source 1..20: mol_type = other DNA
  misc_feature 1..5: note = RNA
  misc_feature 6..15: note = DNA
  misc_feature 16..20: note = RNA
aatctctttactgatataat

SEQ ID NO: 143 - DNA, 20 bp, synthetic construct
  source 1..20: mol_type = other DNA
  misc_feature 1..5: note = RNA
  misc_feature 6..15: note = DNA
  misc_feature 16..20: note = RNA
taatctctttactgatataa

SEQ ID NO: 144 - DNA, 20 bp, synthetic construct
  source 1..20: mol_type = other DNA
  misc_feature 1..5: note = RNA
  misc_feature 6..15: note = DNA
  misc_feature 16..20: note = RNA
aattaatctctttactgata

SEQ ID NO: 145 - DNA, 20 bp, synthetic construct
  source 1..20: mol_type = other DNA
  modified_base 1: mod_base = OTHER; note = 2'-O-methylguanosine phophorothioate
  modified_base 2: mod_base = OTHER; note = 2'-O-methyluridine phophorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methylguanosine
  modified_base 6: mod_base = OTHER; note = Thymidine phosphorothioate
  modified_base 7: mod_base = OTHER; note = Thymidine phosphorothioate
  modified_base 8: mod_base = OTHER; note = Cytidine phosphorothioate
  modified_base 9: mod_base = OTHER; note = Adenosine phosphorothioate
  modified_base 10: mod_base = OTHER; note = Thymidine phosphorothioate
  modified_base 11: mod_base = OTHER; note = Adenosine phosphorothioate
  modified_base 12: mod_base = OTHER; note = Adenosine phosphorothioate
  modified_base 13: mod_base = OTHER; note = Adenosine phosphorothioate
  modified_base 14: mod_base = OTHER; note = Cytidine phosphorothioate
  modified_base 15: mod_base = OTHER; note = Cytidine phosphorothioate
  modified_base 16: mod_base = OTHER; note = 2'-O-methyluridine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methyladenosine phophorothioate
  modified_base 20: mod_base = OTHER; note = 2'-O-methylcytidine phophorothioate
  misc_feature 1..5: note = RNA
  misc_feature 6..15: note = DNA
  misc_feature 16..20: note = RNA
gtcagttcataaacctggac

SEQ ID NO: 146 - protein, 16 aa, synthetic construct
  VARIANT 5: note = Arg or Lys
  VARIANT 6: note = Arg or Lys
  VARIANT 7: note = Arg or Lys
  VARIANT 8: note = Arg or Lys
  VARIANT 9: note = Arg or Lys
  VARIANT 10: note = Arg or Lys
  VARIANT 11: note = Arg or Lys
  VARIANT 12: note = Arg or Lys
  VARIANT 13: note = Arg or Lys
  VARIANT 14: note = Arg or Lys
  VARIANT 15: note = Arg or Lys
  VARIANT 16: note = Arg or Lys
  VARIANT 1..4: note = This region may encompass 1-4 residues
  VARIANT 5..16: note = This region may encompass 3-12 residues
GGGGXXXXXXXXXXXX

SEQ ID NO: 147 - protein, 4 aa, synthetic construct
  VARIANT 1..4: note = This sequence may encompass 1-4 residues
GGGG

SEQ ID NO: 148 - DNA, 105 bp, synthetic construct
  source 1..105: mol_type = other DNA
  misc_difference 1..105: note = This sequence may encompass 5-35 cag repeating units
cagcagcagcagcagcagcagcagcagcagcagcagcagcagcagcagcagcagcagcagcagcagcagcagcagcagcagcagcagcagcagcagcagcagcag

SEQ ID NO: 149 - protein, 16 aa, synthetic construct
  VARIANT 1..4: note = This region may encompass 1-4 residues
  VARIANT 5..16: note = Any standard amino acid or absent, provided that at least three residues are present and are selected from Arg or Lys
GGGGXXXXXXXXXXXX

SEQ ID NO: 150 - protein, 13 aa, synthetic construct
  VARIANT 5: note = Arg or Lys
  VARIANT 6: note = Arg or Lys
  VARIANT 7: note = Arg or Lys
  VARIANT 8: note = Arg or Lys
  VARIANT 9: note = Arg or Lys
  VARIANT 10: note = Arg or Lys
  VARIANT 11: note = Arg or Lys
  VARIANT 12: note = Arg or Lys
  VARIANT 13: note = Arg or Lys
  VARIANT 1..4: note = This region may encompass 1-4 residues
  VARIANT 5..13: note = This region may encompass 7-9 residues
GGGGXXXXXXXXX

SEQ ID NO: 151 - protein, 13 aa, synthetic construct
  VARIANT 5..13: note = This region may encompass 7 or 9 residues
GGGGRRRRRRRRR

SEQ ID NO: 152 - protein, 9 aa, synthetic construct
  VARIANT 1..9: note = This sequence may encompass 7-9 residues
RRRRRRRRR

SEQ ID NO: 153 - protein, 9 aa, synthetic construct
  SITE 1: note = D-Arg
  SITE 2: note = D-Arg
  SITE 3: note = D-Arg
  SITE 4: note = D-Arg
  SITE 5: note = D-Arg
  SITE 6: note = D-Arg
  SITE 7: note = D-Arg
  SITE 8: note = D-Arg
  SITE 9: note = D-Arg
  VARIANT 1..9: note = This sequence may encompass 7-9 residues
RRRRRRRRR